(19) United States
(12) Patent Application Publication
(10) Pub. No.: US 2022/0313428 A1
(43) Pub. Date: Oct. 6, 2022

(54) Frame Features for Transcatheter Mitral Valve Replacement Device

(71) Applicant: Cephea Valve Technologies, Inc., Santa Clara, CA (US)

(72) Inventor: Cathy Bergin, Hugo, MN (US)

(73) Assignee: Cephea Valve Technologies, Inc., Santa Clara, CA (US)

(21) Appl. No.: 17/712,290

(22) Filed: Apr. 4, 2022

Related U.S. Application Data
(60) Provisional application No. 63/171,148, filed on Apr. 6, 2021.

(51) Int. Cl. A61F 2/24

(52) U.S. Cl. CPC A61F 2/2418; A61F 2/2433

(57) ABSTRACT

A prosthetic heart valve includes an outer frame having an atrial portion, a ventricle portion, a narrowed waist portion between the atrial portion and the ventricle portion, and a plurality of outer coupling arms having a first end coupled to the outer frame and a second free end. An inner frame is positioned radially inward of the outer frame and includes a plurality of inner coupling arms having a first end coupled to the inner frame and a second free end, the first ends of the inner coupling arms coupled to the inner frame at a location substantially equidistant between an inflow end of the inner frame and an outflow end of the inner frame. The second free ends of the outer coupling arms are coupled to the second free ends of the inner coupling arms to couple the outer frame to the inner frame.

### CROSS-REFERENCE TO RELATED APPLICATIONS

This applications claims priority to the filing date of U.S. Provisional Patent Application No. 63/171,148, filed Apr. 6, 2021, the disclosure of which is hereby incorporated by reference herein.

### BACKGROUND OF THE DISCLOSURE

Heart valve disease is a significant cause of morbidity and mortality. A primary treatment of this disease is valve replacement. One form of replacement device is a bioprosthetic valve. Collapsing these valves to a smaller size or into a delivery system enables less invasive delivery approaches compared to conventional open-chest, open-heart surgery. Collapsing the implant to a smaller size and using a smaller delivery system minimizes the access site size and reduces the number of potential periprocedural complications.

The size to which an implant can be collapsed is limited by the volume of materials used in the implant, the strengths and shapes of those materials, and the need to function after re-expansion.

### BRIEF SUMMARY

According to one aspect of the disclosure, a prosthetic heart valve includes a collapsible and expandable outer frame configured to engage tissue of a native heart valve. The outer frame has an atrial portion adapted to be positioned on an atrial side of the native heart valve, a ventricle portion adapted to be positioned on a ventricle side of the native heart valve, a narrowed waist portion between the atrial portion and the ventricle portion, and a plurality of outer coupling arms having a first end coupled to the outer frame and a second free end. A collapsible and expandable inner frame is positioned radially inward of the outer frame, the inner frame including a plurality of inner coupling arms having a first end coupled to the inner frame and a second free end, the first ends of the inner coupling arms coupled to the inner frame at a location substantially equidistant between an inflow end of the inner frame and an outflow end of the inner frame. A prosthetic valve assembly is coupled to, and positioned radially inward of, the inner frame. The second free ends of the outer coupling arms are coupled to the second free ends of the inner coupling arms to couple the outer frame to the inner frame.

According to another aspect of the disclosure, a prosthetic heart valve includes a collapsible and expandable outer frame configured to engage tissue of a native heart valve. The outer frame has an atrial portion adapted to be positioned on an atrial side of the native heart valve, a ventricle portion adapted to be positioned on a ventricle side of the native heart valve, a narrowed waist portion between the atrial portion and the ventricle portion, and a plurality of outer coupling arms having a first end coupled to the outer frame and a second free end. A collapsible and expandable inner frame is positioned radially inward of the outer frame. The inner frame has a first row of generally diamond shaped cells at an inflow end of the inner frame, and a second row of generally diamond shaped cells at an outflow end of the inner frame. The inner frame includes a plurality of inner coupling arms having a first end coupled to the inner frame and a second free end, the second free ends of the outer coupling arms being coupled to the second free ends of the inner coupling arms to couple the outer frame to the inner frame. A prosthetic valve assembly is coupled to, and positioned radially inward of, the inner frame. The inner frame includes a plurality of axial struts extending from the second row of generally diamond shaped cells in a direction away from the inflow end of the inner frame. The axial struts define commissure windows, prosthetic leaflets of the prosthetic valve assembly being coupled to the axial struts via the commissure windows, a plurality of support struts coupling the axial struts to the second row of generally diamond shaped cells.

According to a further aspect of the disclosure, a prosthetic heart valve includes a collapsible and expandable outer frame configured to engage tissue of a native heart valve. The outer frame has an atrial portion adapted to be positioned on an atrial side of the native heart valve, a ventricle portion adapted to be positioned on a ventricle side of the native heart valve, a narrowed waist portion between the atrial portion and the ventricle portion, and a plurality of outer coupling arms having a first end coupled to the outer frame and a second free end. A collapsible and expandable inner frame is positioned radially inward of the outer frame. The inner frame includes a plurality of inner coupling arms having a first end coupled to the inner frame and a second free end, the second free ends of the outer coupling arms being coupled to the second free ends of the inner coupling arms to couple the outer frame to the inner frame. A prosthetic valve assembly is coupled to, and positioned radially inward of, the inner frame. The outer frame includes a first row of cells at an inflow end of the outer frame, and a second row of cells at an outflow end of the outer frame, the first row of cells and the second row of cells having the same number of cells.

### BRIEF DESCRIPTION OF THE DRAWINGS

FIG. 1A is a side view of an assembled stent frame of a prosthetic heart valve of the prior art, the stent frame being shown in an expanded condition.

FIG. 1B is a side view of an outer frame of the stent frame of FIG. 1A.

FIG. 1C is a flattened view of the outer stent of FIG. 1B, as if cut longitudinally and laid out flat on a table in an unexpanded condition.

FIG. 1D is a side view of an inner frame of the stent frame of FIG. 1A.

FIG. 1E is a flattened view of the inner stent of FIG. 1D, as if cut longitudinally and laid out flat on a table in an unexpanded condition.

FIG. 2A is a side view of an assembled stent frame of a prosthetic heart valve according to an embodiment of the disclosure, the stent frame being shown in an expanded condition.

FIG. 2B is a side view of an outer frame of the stent frame of FIG. 2A.

FIG. 2C is a flattened view of the outer stent of FIG. 2B, as if cut longitudinally and laid out flat on a table in an unexpanded condition.

FIG. 2D is a side view of an inner frame of the stent frame of FIG. 2A.

FIG. 2E is a flattened view of the inner stent of FIG. 2D, as if cut longitudinally and laid out flat on a table in an unexpanded condition.

FIG. 3A is a side view of an outer frame of a stent of a prosthetic heart valve in an expanded condition according to another embodiment of the disclosure.

FIG. 3B is an enlarged view of an atrial tip portion of the outer frame of FIG. 3A.

FIG. 3C is a flattened view of the outer stent of FIG. 3A, as if cut longitudinally and laid out flat on a table in an unexpanded condition.

FIG. 3D is an enlarged view of the atrial tip portion of the outer frame of FIG. 3C in the unexpanded condition.

FIGS. 3E-F are perspective views from the atrial and ventricular sides, respectively, of a prosthetic heart valve incorporating the outer frame of FIG. 3A.

FIG. 3G is a view of an interior of the prosthetic heart valve of FIGS. 3E-F in the collapsed condition.

FIG. 3H is a side view of the prosthetic heart valve of FIGS. 3E-F being deployed from a delivery device.

FIG. 4A1 is a flattened view of a portion of an outer frame of a prosthetic heart valve, the portion of the outer frame being shown as if the outer frame was cut longitudinally and laid flat on a table in an unexpanded condition.

FIGS. 4A2-4A3 illustrate different geometries at a stent node.

FIG. 4B is a cut-away side view of the outer frame of FIG. 4A1 coupled to an inner frame in an assembled and expanded condition.

FIG. 4C is a cut-away side view of the outer frame of FIG. 4A1 coupled to an inner frame in an assembled and expanded condition.

FIGS. 4D-F are perspective, side, and top views, respectively of the inner and outer frames of FIG. 4C assembled and expanded.

FIG. 5A is a side view of an inner frame in an unexpanded condition, according to another aspect of the disclosure.

FIG. 5B is a side view of the inner frame of FIG. 5A, shown in both the unexpanded and expanded conditions, with coupling arms set to a first shape.

FIG. 5C is a side view of the inner frame of FIG. 5A, shown in an expanded condition, with coupling arms set to a second shape different than the first shape of FIG. 5B.

FIGS. 6A-B are perspective and side views, respectively, of another embodiment of an outer frame of a prosthetic heart valve.

FIG. 6C is a flattened view of the outer frame of FIGS. 6A-B, as if cut longitudinally and laid out flat on a table in an unexpanded condition.

FIGS. 6D-E are perspective and side views, respectively, of another embodiment of an inner frame of a prosthetic heart valve, which may be configured for use with the outer frame of FIGS. 6A-C.

FIG. 6F is a flattened view of the inner frame of FIGS. 6D-E, as if cut longitudinally and laid out flat on a table in an unexpanded condition.

FIGS. 7A-C are schematic illustrations showing a portion of the inner frame of FIGS. 6D-E coupled to different sized outer frames.

FIG. 8A shows four MicroCT images, taken at 90 degree rotations, of an interior of the prosthetic heart valve of FIGS. 6A-F in a collapsed condition within a delivery device.

FIG. 8B is a side view of the prosthetic heart valve of FIGS. 6A-F being deployed from a delivery device.

### DETAILED DESCRIPTION

As used herein, the term “inflow end,” when used in connection with a prosthetic heart valve, refers to an end of the prosthetic heart valve into which blood first flows when the prosthetic heart valve is implanted in an intended position and orientation. On the other hand, the term “outflow end,” when used in connection with a prosthetic heart valve, refers to the end of the prosthetic heart valve through which blood exits when the prosthetic heart valve is implanted in an intended position and orientation. In the figures, like numbers refer to like or identical parts. As used herein, the terms “substantially,” “generally,” “approximately,” and “about” are intended to mean that slight deviations from absolute are included within the scope of the term so modified. When ranges of values are described herein, those ranges are intended to include sub-ranges. For example, a recited range of 1 to 10 includes 2, 5, 7, and other single values, as well as all sub ranges within the range, such as 2 to 6, 3 to 9, 4 to 5, and others.

The present disclosure is generally directed to collapsible prosthetic mitral valves, and in particular various features of stents thereof to provide enhanced functionality. However, it should be understood that the features described herein may apply to other types of prosthetic heart valves, including prosthetic heart valves that are adapted for use in other heart valves, such as the tricuspid heart valve. Further, the features of the prosthetic heart valves described herein may, in some circumstances, be suitable for surgical (e.g. non-collapsible) prosthetic heart valves. However, as noted above, the disclosure is provided herein in the context of a collapsible and expandable prosthetic mitral valve.

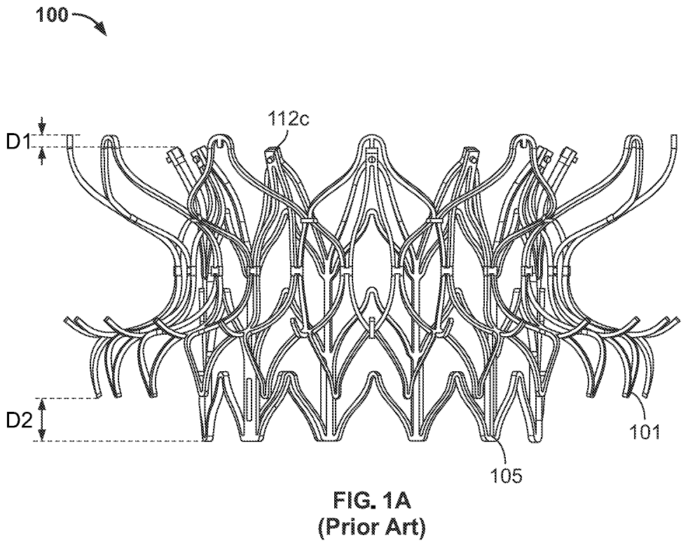

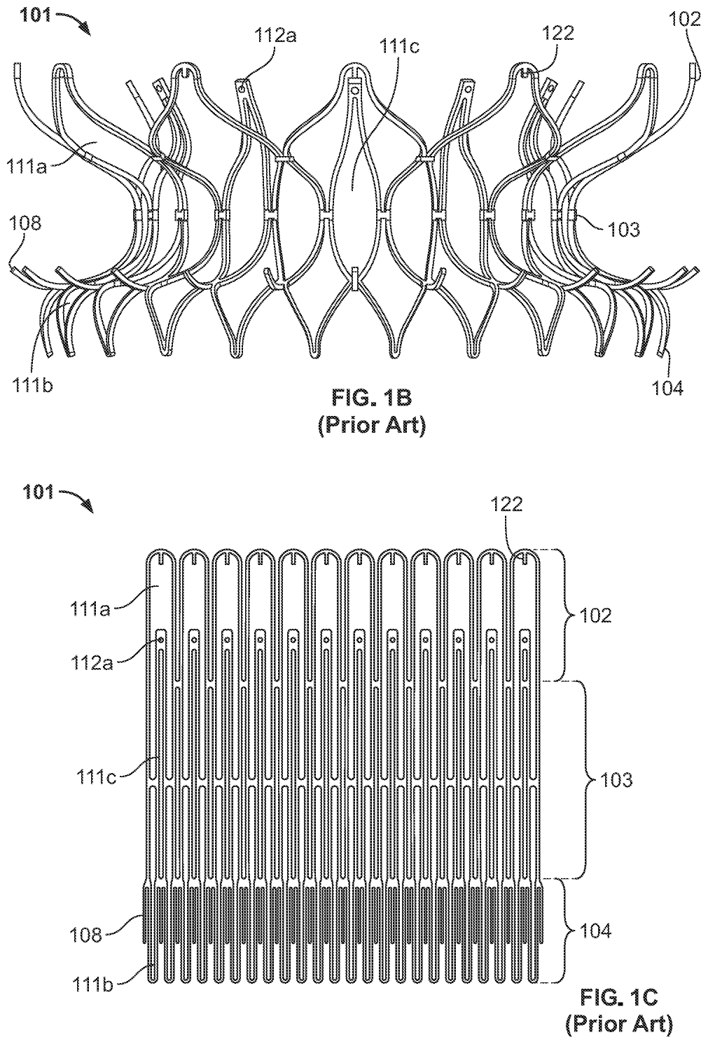

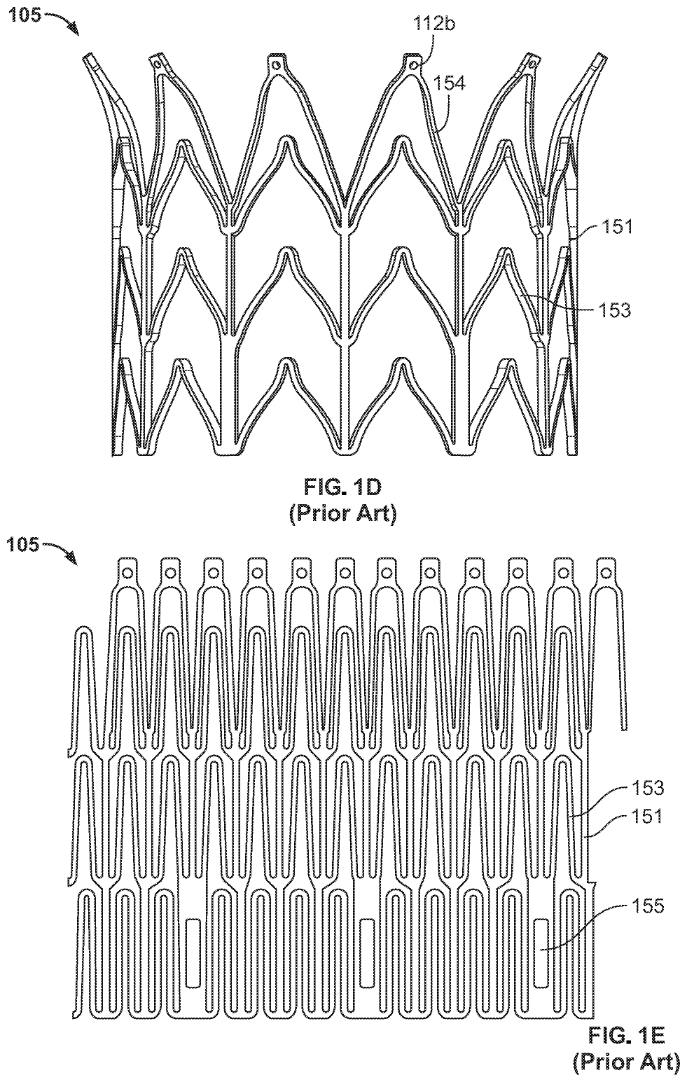

FIG. 1A illustrates an example of a collapsible and expandable prosthetic heart valve 100, according to the prior art, which may be particularly suited for replacement of a native mitral or tricuspid valve. It should be understood that the prosthetic heart valve 100 illustrated in FIG. 1A omits certain features that would typically be included, such as a valve assembly to assist in controlling blood flow through the prosthetic heart valve, and interior and/or exterior fabrics or tissue skirts to assist with providing a seal around the prosthetic heart valve and/or with enhancing tissue ingrowth to fix the prosthetic heart valve within the native heart valve over time. However, for purposes of simplicity, the prosthetic leaflet(s) and skirt(s) are omitted from the drawings for clarity of illustration.

The prosthetic heart valve 100 is illustrated in FIG. 1A in an expanded configuration. The stent of the prosthetic heart valve 100 may include an outer stent or frame 101 and an inner stent or frame 105 positioned radially within the outer frame. The outer frame 101 may be primarily for anchoring the prosthetic heart valve 100 within the native heart valve annulus, while the inner frame 105 may be primarily for holding the prosthetic valve assembly in the desired position and orientation.

Outer frame 101 is illustrated in FIGS. 1B-C isolated from other components of the prosthetic heart valve 100. In FIG. 1B, the outer frame 101 is illustrated in an expanded condition. In FIG. 1C, the outer frame 101 is illustrated in an unexpanded condition, as if cut longitudinally and laid flat on a table. As used herein, the term “unexpanded” refers to the state of the stent prior to being shape-set (e.g. immediately after it is cut from a tube of nitinol). After being formed, the stent may be shape-set to the expanded condition, and may also have a collapsed condition in which the stent is collapsed to a smaller size from its expanded condition. The shape of the stent may be similar, but not necessarily identical, in the unexpanded and collapsed conditions. As shown in FIGS. 1B-C, outer frame 101 may include an atrial portion or anchor 102, a ventricular portion or anchor 104, and a central portion 103 coupling the atrial portion to the ventricular portion. The central portion 103 may be between atrial portion 102 and ventricular portion 104. Atrial portion 102 may be configured and adapted to be disposed on an atrial side of a native valve annulus, and may flare radially outwardly from the central portion 103. Ventricular portion 104 may be configured and adapted to be disposed on a ventricle side of the native valve annulus, and may also flare radially outwardly from the central portion 103. The central portion 103 may be configured to be situated in the valve orifice, for example in contact with the native valve annulus. In use, the atrial portion 102 and ventricle portion 104 effectively clamp the native valve annulus on the atrial and ventricular sides thereof, respectively, holding the prosthetic heart valve 100 in place.

The atrial portion 102 may be formed as a portion of a stent or other support structure that includes or is formed by a plurality of generally diamond shaped cells, although other suitable cell shapes, such as triangular, quadrilateral, or polygonal may be appropriate. In some examples, the atrial portion 102 may be formed as a braided mesh, as a portion of a unitary stent, or a combination thereof. According to one example, the stent that includes the atrial portion 102 may be laser cut from a tube of nitinol and heat set to a desired shape so that the stent, including atrial portion 102, is collapsible for delivery, and re-expandable to the set-shape during deployment. The atrial portion 102 may be heat set into a suitable shape to conform to the native anatomy of the valve annulus to help provide a seal and/or anchoring between the atrial portion 102 and the native valve annulus. The heat-set atrial portion 102 may be partially or entirely covered by a cuff or skirt, on the luminal and/or abluminal surface of the atrial portion 102. The skirt may be formed of any suitable material, including biomaterials such as bovine pericardium, biocompatible polymers such as ultra-high molecular weight polyethylene, woven polyethylene terephthalate (“PET”) or expanded polytetrafluoroethylene (“ePTFE”), or combinations thereof. The atrial portion 102 may include features for connecting the atrial portion to a delivery system. For example, the atrial portion 102 may include pins or tabs 122 around which sutures (or suture loops) of the delivery system may wrap, so that while the suture loops are wrapped around the pins or tabs 122, the outer frame 101 maintains a connection to the delivery device.

The ventricular portion 104 may also be formed as a portion of a stent or other support structure that includes or is formed of a plurality of diamond shaped cells, although other suitable cell shapes, such as triangular, quadrilateral, or polygonal may be appropriate. In some examples, the ventricular portion 104 may be formed as a braided mesh, as a portion of a unitary stent, or a combination thereof. According to one example, the stent that includes the ventricular portion 104 may be laser cut from a tube of nitinol and heat set to a desired shape so that the ventricular portion 104 is collapsible for delivery, and re-expandable to the set-shape during deployment. The ventricular portion 104 may be partially or entirely covered by a cuff or skirt, on the luminal and/or abluminal surface of the ventricular portion 104. The skirt may be formed of any suitable material described above in connection with the skirt of atrial portion 102. It should be understood that the atrial portion 102 and ventricular portion 104 may be formed as portions of a single support structure, such as a single stent or braided mesh. However, in other embodiments, the atrial portion 102 and ventricular portion 104 may be formed separately and coupled to one another.

As illustrated in FIG. 1A, the inner frame 105 may be positioned radially within the outer frame 101 when the inner and outer frames are assembled together. Inner frame 105 is illustrated in FIGS. 1D-E isolated from other components of the prosthetic heart valve 100. In FIG. 1D, the inner frame 105 is illustrated in an expanded condition. In FIG. 1E, the inner frame 105 is illustrated in an unexpanded condition, as if cut longitudinally and laid flat on a table. As shown in FIGS. 1D-E, the inner frame 105 may include a plurality of axially or longitudinally extending struts 151 and interconnecting v-shaped strut members 153. According to some embodiments, the inner frame 105 may have more or fewer v-shaped members 153 extending circumferentially around the diameter thereof than the number of cells in the atrial portion 102 and/or ventricular portion 104 of the outer frame 101, such as double or half the number. In some examples, the inner frame 105 may flare radially outwards at the atrial end, e.g., to conform to the flare of the atrial portion 102 of the outer frame 101. One or more prosthetic leaflets may be coupled to the inner frame 105 to form a prosthetic valve assembly, the prosthetic valve assembly configured to allow unidirectional flow of blood through the prosthetic valve assembly from the atrial end toward the ventricular end of the prosthetic heart valve 100. As best illustrated in FIG. 1E, the inner frame 105 may include a plurality of commissure windows 155 formed in axial struts 151. For example, inner frame 105 may include three generally rectangular shaped commissure windows 155 equidistantly spaced around the circumference of the inner frame, with each commissure window adapted to provide a location for coupling two adjacent prosthetic leaflets to the axial strut 151. However, more or fewer commissure windows 155 may be provided depending on how many prosthetic leaflets will be coupled to the inner frame 105.

The outer frame 101 and/or the inner frame 105 may be formed of a superelastic and/or shape memory material such as nitinol. According to some examples, other biocompatible metals and metal alloys may be suitable. For example, superelastic and/or self-expanding metals other than nitinol may be suitable, while still other metals or metal alloys such as cobalt chromium or stainless steel may be suitable, particularly if the stent or support structure is intended to be balloon expandable. In some examples, the outer frame 101 and/or inner frame 105 may be laser cut from one or more tubes, such as a shape memory metal tube. The shape memory metal tube may be nitinol or any other bio-compatible metal tube. For example, the outer frame 101 may be laser cut from a first tube while the inner frame 105 may be laser cut from a second tube of smaller diameter.

The prosthetic heart valve 100 may be adapted to expand from a collapsed or constrained configuration to an expanded configuration. According to some examples, the prosthetic heart valve 100 may be adapted to self-expand, although the prosthetic heart valve could instead be partially or fully expandable by other mechanisms, such as by balloon expansion. The prosthetic heart valve 100 may be maintained in the collapsed configuration during delivery, for example via one or more overlying sheaths that restrict the valve from expanding. The prosthetic heart valve 100 may be expanded during deployment from the delivery device once the delivery device is positioned within or adjacent the native valve annulus. In the expanded configuration, the atrial portion 102 and ventricular portion 104 may extend radially outward from a central longitudinal axis of the prosthetic heart valve 100 and/or central portion 103, and may be considered to flare outward relative to the central longitudinal axis of the replacement valve and/or central portion 103. The atrial portion 102 and ventricular portion 104 may be considered flanged relative to central portion 103. The flared configuration of atrial and ventricular portions 102, 104 relative to central portion 103 is described in the context of a side view of the outer frame 101, as can be best seen in FIG. 1B. In some embodiments, the flared configuration of the atrial and ventricular portions 102, 104 and the central portion 103 may define a general hour-glass shape in a side view of the outer frame 101. That is, the atrial and ventricular portions 102, 104 may be flared outwards relative to the central portion 103 and then curved or bent to point at least partially back in the axial direction. It should be understood, however, that an hour-glass configuration is not limited to symmetrical configuration.

The outer frame 101 may be configured to expand circumferentially (and radially) and foreshorten axially as the prosthetic heart valve 100 expands from the collapsed delivery configuration to the expanded deployed configuration. As described herein, the outer frame 101 may define a plurality of atrial cells 111a in one circumferential row and a plurality of ventricular cells 111b in another circumferential row. Each of the plurality of cells 111a, 111b may be configured to expand circumferentially and foreshorten axially upon expansion of the outer frame 101. As shown, the cells 111a-b may each be diamond-shaped. In the illustrated embodiment, the outer frame 101 includes twelve atrial cells 111a, and twenty-four ventricular cells 111b. In addition, a third plurality of cells 111c in another circumferential row. Cells 111c may have a first end that is within a corresponding atrial cell 111a, at least when the frame is collapsed (similar to the unexpanded condition shown in FIG. 1C). Cells 111c may have a second end that is positioned between pairs of adjacent ventricular cells 111b, at least when the frame is collapsed (similar to the unexpanded condition shown in FIG. 1C). In this particular example, the outer frame 101 includes twelve center cells 111c.

Still referring to FIGS. 1B-C, a pin or tab 122 may extend from an apex of each atrial cell 111a in a direction toward the outflow end of the outer frame 101. Although one pin or tab 122 is illustrated in each atrial cell 111a, in other embodiments fewer than all of the atrial cells may include a pin or tab. Each center cell 111c may include an aperture 112a or other coupling feature at a first apical end thereof for coupling to the inner frame 105, as is described in greater detail below. In the illustrated embodiment, the aperture 112a is positioned at the inflow apex of center cells 111c, and each center cell includes an aperture, although in other embodiments fewer than all of the cells may include such apertures. In the expanded condition of the outer frame 101, as shown in FIG. 1B, the apex of the center cells 111c that include the apertures 112a may be positioned radially inwardly of the apex of the atrial cells 111a near the inflow end of the outer frame. In addition, each center cell 111c may include a tine or barb 108 extending from the opposite apex on the outflow end of the center cell, although fewer than all of the center cells may include such barbs. In the collapsed condition of the outer frame 101 (similar to the unexpanded condition shown in FIG. 1C), each barb 108 extends toward the outflow end of the outer frame, each barb being positioned between two adjacent ventricular cells 111b. In the expanded condition of the outer frame 101, as shown in FIG. 1B, the barbs 108 may hook upwardly back toward the inflow end, the barbs being configured to pierce native tissue of the valve annulus, such as the native leaflets, to help keep the prosthetic heart valve from migrating under pressure during beating of the heart. Typically, the term “tine” may refer to a structure configured to pierce into tissue, while the term “barb” may refer to a tine that also includes a barb-like structure to prevent the barb from pulling out of the tissue once pierced. However, as used herein, the term “barb” includes tines, with or without actual “barb”-like structures that prevent pulling out of tissue, unless specifically noted otherwise.

The inner frame 105 may be configured to expand circumferentially (and radially) while maintaining the same (or about the same) axial dimension (e.g., be non-foreshortening) as the prosthetic heart valve 100 expands from the collapsed delivery configuration to the expanded configuration. The axial struts 151 may contribute to this non-foreshortening functionality. By being non-foreshortening, the inner frame 105 may prevent (or reduce) strain from being placed on the prosthetic leaflets when the inner frame 105 transitions between the collapsed and expanded conditions. Thus, while the outer frame 101 may be designed to be foreshortening, the inner frame 105 may be designed so as to be substantially non-foreshortening.

Inner frame 105 may include twelve longitudinal struts 151, with three rows of twelve v-shaped members 153. However, in other embodiments, more or fewer longitudinal struts 151 may be included, and more or fewer rows of v-shaped members 153 may be included. In the illustrated embodiment, the number of longitudinal struts 151 is equal to the number of atrial cells 111a of the outer frame 101. In addition, v-shaped coupling members 154 may extend from each adjacent pair of longitudinal struts 151. These v-shaped coupling members 154 may have half-diamond shapes, with the apex of each half-diamond shape including an aperture 112b, the v-shaped coupling members generally flaring radially outwardly in the expanded condition of inner frame 105.

Referring back to FIG. 1A, in the expanded conditions of the outer frame 101 and the inner frame 105, the top portion of the center cells 111c may flare outwardly with a contour that substantially matches the outward flare of the v-shaped coupling members 154, so that apertures 112a and 112b align with each other. A coupling member, such as a rivet 112c, may pass through apertures 112a and 112b to couple the outer frame 101 to the inner frame 105.

Additional features and example replacement valves may be described in International patent application publication WO/2018/136959, filed Jan. 23, 2018, and titled “REPLACEMENT MITRAL VALVES,” which is hereby incorporated by reference herein.

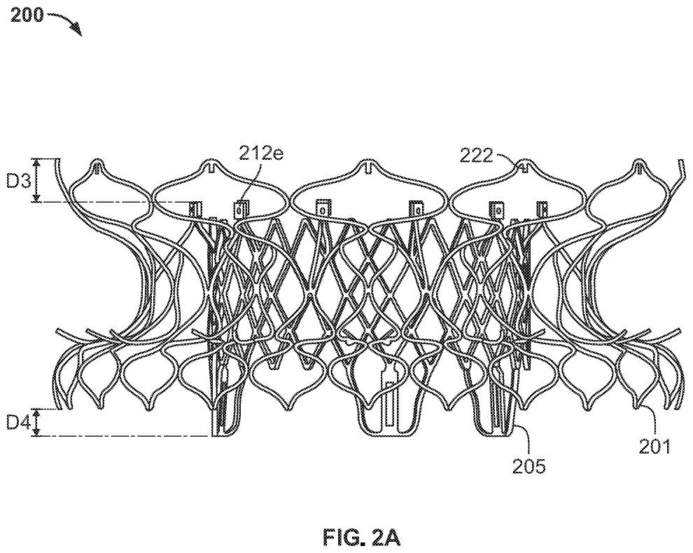

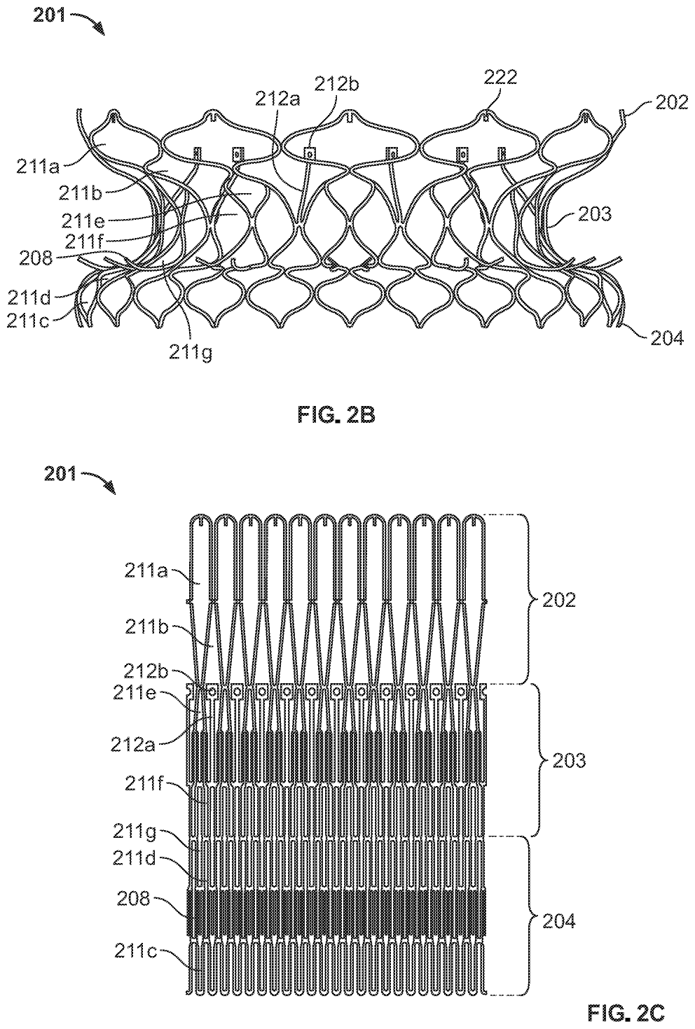

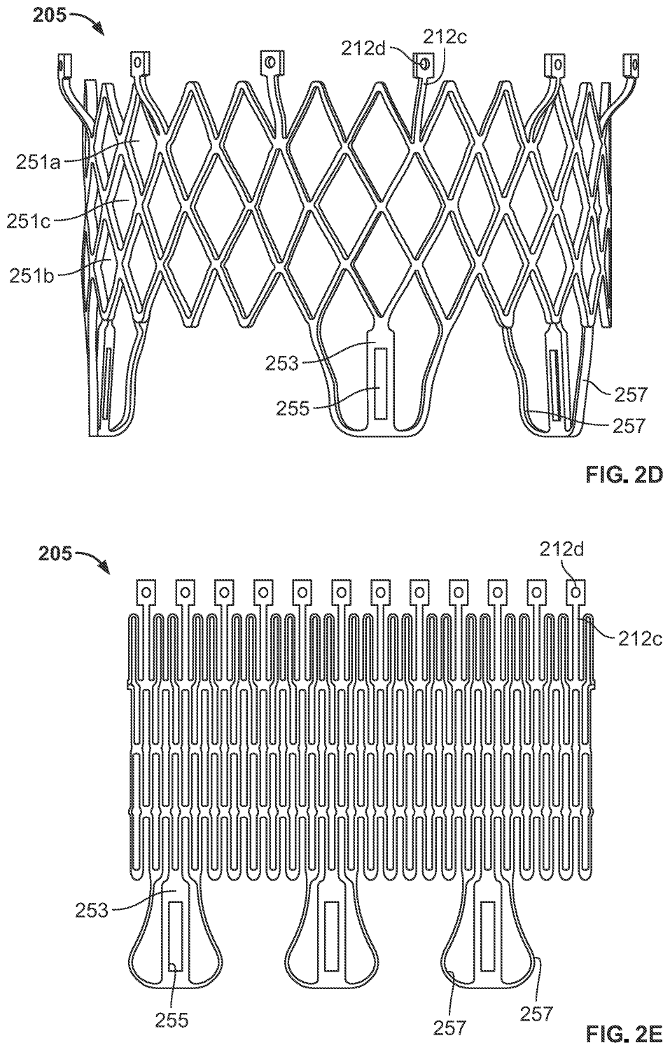

FIG. 2A illustrates another embodiment of a collapsible and expandable prosthetic heart valve 200, which may be particularly suited for replacement of a native mitral or tricuspid valve. The overall general structure of prosthetic heart valve 200 may be substantially similar to that of prosthetic heart valve 100 in both structure and function, but prosthetic heart valve 200 may have various differences to provide for certain benefits compared to prosthetic heart valve 100. For the purpose of brevity, only the differences between prosthetic heart valve 200 compared to prosthetic heart valve 100 are described in detail below, with the remaining features of prosthetic heart valve 200 being similar or identical to the corresponding features of prosthetic heart valve 100. As with prosthetic heart valve 100, it should be understood that the prosthetic heart valve 200 illustrated in FIG. 2A omits certain features such as prosthetic leaflets and luminal and/or abluminal stent skirts. The prosthetic heart valve 200 is illustrated in FIG. 2A in an expanded configuration. The stent of the prosthetic heart valve 200 may include an outer stent or frame 201 and an inner stent or frame 205 positioned radially within the outer frame.

Outer frame 201 is illustrated in FIGS. 2B-C isolated from other components of the prosthetic heart valve 200. In FIG. 2B, the outer frame 201 is illustrated in an expanded condition. In FIG. 2C, the outer frame 201 is illustrated in an unexpanded condition, as if cut longitudinally and laid flat on a table. Similar to outer frame 101, outer frame 201 may include an atrial portion or anchor 202, a ventricular portion or anchor 204, and a central portion 203 coupling the atrial portion to the ventricular portion.

The outer frame 201 may be configured to expand circumferentially (and radially) and foreshorten axially as the prosthetic heart valve 200 expands from the collapsed delivery configuration to the expanded deployed configuration. The outer frame 201 may define a plurality of atrial cells 211a, 211b in two circumferential rows. For example, the first row of atrial cells 211a may be generally diamond shaped and positioned on the inflow end of the outer frame 201. The second row of atrial cells 211b may be positioned at least partially between adjacent atrial cells 211a in the first row, with the atrial cells 211b in the second row being positioned farther from the inflow end than the first row of atrial cells 211a. The outer stent 201 may include twelve atrial cells 211a in the first row each having a diamond-shape, and twelve atrial cells 211b in the second row each having a skewed diamond shape. This skewed diamond shape, which is wider nearer the inflow (or top) end and narrower nearer the outflow (or bottom) end, may assist in transitioning from twelve cells per row on the atrial side of the stent to twenty-four cells per row on the ventricular side.

The outer frame 201 may include a plurality of ventricular cells 111c in a first row, and another plurality of ventricular cells 11d in a second row. The first row of ventricular cells 211c may be at the outflow end of the outer frame 201, and the second row of ventricular cells 211d may be positioned farther from the outflow end than, and adjacent to, the first row of ventricular cells 211c. In the illustrated embodiment the first and second rows of ventricular cells 211c, 211d are all generally diamond-shaped and have substantially the same, or an identical, size, with twenty-four cells in the first row of ventricular cells 211c and twenty-four cells in the second row of ventricular cells 211d.

Outer stent 201 is also illustrated as including three rows of center cells. A first row of center cells 211e is positioned adjacent the atrial end of the outer stent 201, each cell 211e being positioned between a pair of adjacent atrial cells 211b. Each center cell 211e may be substantially diamond-shaped, but it should be understood that adjacent center cells 211e do not directly touch one another. The first row of center cells 211e may include twelve center cells 211e, with the combination of atrial cells 211b and the center cells 211e helping transition from rows of twelve cells on the atrial side to rows of twenty-four cells on the ventricular side. A second row of center cells 211f may be positioned at a longitudinal center of the outer frame 201, each center cell 211f being positioned between an atrial cell 211b and center cell 211e. In the illustrated embodiment, center cells 211f in the second row may be diamond-shaped, with the second row including twenty-four center cells 211f. Finally, a third row of center cells 211g may be positioned between the second row of center cells 211f and the second row of ventricular cells 211d. The third row of center cells 211g may include twenty-four cells and they may each be substantially diamond-shaped.

All of the cells 211a-g may be configured to expand circumferentially and foreshorten axially upon expansion of the outer frame 201. Similar to outer frame 100, a pin or tab 222 may extend from an apex of each atrial cell 211a in the first row in a direction toward the outflow end of the outer frame 201. Although one pin or tab 222 is illustrated in each atrial cell 211a in the first row, in other embodiments fewer than all of the atrial cells in the first row may include a pin or tab. Whereas outer frame 101 included apertures 112a at an apex of a center cell 111c, outer frame 201 may instead include coupling arms 212a. Each coupling arm 212a may be a strut that is coupled to a bottom or outflow apex of each atrial cell 211b in the second row, with each strut extending toward the inflow end of the outer frame 201 to a free end of the coupling arm 212a. The free end of each coupling arm 212a may include an aperture 212b for coupling to the inner frame 205, as described in greater detail below. In the collapsed condition (similar to the unexpanded condition shown in FIG. 2C), each coupling arm 212a is substantially surrounded by an atrial cell 211b in the second row. In the expanded condition, best shown in FIG. 2B, the coupling arms 212a may extend radially inwardly and have a contour so that the free end extends substantially parallel to the center longitudinal axis of the outer frame 201. In addition, outer frame 201 may include a plurality of tines or barbs 208 extending from a center portion or ventricular portion of the outer frame for piercing native tissue in the native annulus or in the native leaflets. In the illustrated embodiment, each barb 208 is connected to a ventricular cell 211d in the second row. In some embodiments, the barb 208 may be coupled to an inflow or outflow apex of each cell. In the particular illustrated embodiment, the barbs 208 are couple to ventricular cells 211d on an inflow half of the cell, on either side of the inflow apex. For example, the barb 208 in one ventricular cell 211d may be coupled to the inflow half of that cell on a right side of the apex, with the adjacent ventricular cell 211d having a barb coupled to the inflow half of that cell on a left side of the apex. With this configuration, the barbs 208 are provided in pairs with relatively little space between the barbs of a pair, but a relatively large space between adjacent pairs. However, it should be understood that the barbs 208 may in other embodiments be centered with even spacing between adjacent barbs, similar to that shown and described in connection with FIG. 1B. In the collapsed condition of the outer frame 201 (similar to the unexpanded condition shown in FIG. 2C), each barb 208 extends toward the outflow end of the outer frame, each barb being positioned within a ventricular cell 211d in the second row. In the expanded condition of the outer frame 201, as shown in FIG. 2B, the barbs 208 may hook upwardly back toward the inflow end, the barbs being configured to pierce native tissue of the valve annulus, such as the native leaflets, to help keep the prosthetic heart valve from migrating under pressure during beating of the heart.

As illustrated in FIG. 2A, the inner frame 205 may be positioned radially within the outer frame 201 when the inner and outer frames are assembled together. Inner frame 205 is illustrated in FIGS. 2D-E isolated from other components of the prosthetic heart valve 200. In FIG. 2D, the inner frame 205 is illustrated in an expanded condition. In FIG. 2E, the inner frame 205 is illustrated in an unexpanded condition, as if cut longitudinally and laid flat on a table. Whereas inner frame 105 includes longitudinal struts 151 and is non-foreshortening, inner frame 205 instead includes a plurality of rows of diamond-shaped cells so that the inner frame 205 foreshortens upon expansion. In the illustrated example, inner frame 205 includes three rows of diamond-shaped cells, including a first row of cells 251a at the inflow end of the inner frame, a second row of cells 251b at the outflow end of the inner frame, and a third row of cells 251c positioned between the first and second rows. In some embodiments, the inner frame 205 may include more or fewer rows of cells. In the expanded condition shown in FIG. 2D, the three rows of cells 251a-c may be substantially cylindrical.

One or more prosthetic leaflets may be coupled to the inner frame 205 to form a prosthetic valve assembly, the prosthetic valve assembly configured to allow unidirectional flow of blood through the prosthetic valve assembly from the atrial end toward the ventricular end of the prosthetic heart valve 200. As illustrated in FIGS. 2D-E, the inner frame 205 may include a plurality of commissure windows 255 formed in axial struts 253 extending from selected cells 251b at the outflow end of the inner frame 205. For example, inner frame 205 may include three generally rectangular shaped commissure windows 255 equidistantly spaced around the circumference of the inner frame, with each commissure window adapted to provide a location for coupling two adjacent prosthetic leaflets to the axial strut 253. However, more or fewer commissure windows 255 may be provided depending on how many prosthetic leaflets will be coupled to the inner frame 205. Additional support struts 257 may connect the axial struts 253 to the cells 251b. In particular, a first support strut 257 may couple the outflow end of each axial strut 253 to the outflow apex of a first cell 251b on a first side of the axial strut, and a second support strut 257 may couple the outflow end of each axial strut 253 to the outflow apex of a second cell 251b on a second opposite side of the axial strut, with the axial strut coupled to a third cell 251b between the first and second cells. As shown in FIGS. 2D-E, the support struts 257 may be contoured so as to avoid presenting any sharp tips, which may help avoid damaging the anatomy.

The inner frame 205 may also include a plurality of coupling arms 212c. Each coupling arm 212c may have a first end coupled to the inner frame 205 at an inflow end of the inner frame. In particular, the first end of each coupling arm 212c may be attached to a junction between two adjacent cells 251a in the first row at the inflow end. The coupling arms 212c may extend in a direction away from the outflow end of the inner frame 205 to a free end, with the free end including an aperture 212d therein. In the expanded condition, as shown in FIG. 2D, the coupling arms 212c may initially extend radially outwardly from the inner frame 205, with the free end being contoured so that the free end extends substantially parallel to the longitudinal axis of the inner frame 205. In the illustrated embodiment, inner frame 205 may include a total of twelve coupling arms 212c spaced equidistantly around the circumference of the inner frame. Preferably, the number of coupling arms 212c corresponds to the number of coupling arms 212a. Referring back to FIG. 2A, a coupling member, such as a suture or a rivet 212e, may pass through apertures 212b and 212d to couple the outer frame 201 to the inner frame 205.

Various differences between prosthetic heart valves 100 and 200 are now described in greater detail. Outer frame 101 may include a relatively small number of cells which each define a relatively large area. For example, outer frame 101 includes only two rows of cells 111a, 111b for anchoring (which excludes the row of cells 111c which in large part serve to connect the outer frame 101 to the inner frame 105). On the other hand, despite having a generally similar profile as outer frame 101, outer frame 201 includes a larger number of cells that typically define a smaller area. For example, outer frame 201 may be thought of as including six rows of full cells (if cells 211b and 211e are counted as a single row considering the circumferential overlap between those cells). One result of this difference is that the outer frame 101 may have relatively little redundancy compared to outer frame 201. Thus, in the event that a strut defining a cell (or a portion thereof) fractures, the likelihood of stent failure (or the likely detrimental effect of a failure) may be significantly reduced in outer stent 201 compared to outer stent 101, due to increased redundancy in the design. Another benefit of the increased number of cells in outer stent 201 compared to outer stent 101 concerns any tissue and/or fabric skirts coupled to the outer stent 201. For example, due to additional stent structure, more options may be available for how and where to attach tissue and/or fabric skirts to the outer stent 201. This additional stent structure may also better distribute the pressure applied by outer stent 201 to the patient's tissue, thereby reducing the risk of tissue erosion. Still further, the greater number of cells, and smaller area of cells, of outer stent 201 compared to outer stent 101, may allow for reduced forces required to collapse the outer stent 201 during loading into a delivery device, and also reduce forces experienced during deploying the outer stent. This may result in lower strain experienced by the outer stent 201, compared to outer stent 101, and thus improve durability of the outer stent 201. Stated in another way, having more cells with a desired aspect ratio may allow for each cell to have a relatively small strut width, while still being able to maintain a desired stiffness. The diamond cell pattern may allow for the cells (and the stent) to collapse without significant twisting or torsion. This type of twisting or torsion, which may be a primary driver of higher strain, may be more likely to occur in outer stent 101 compared to outer stent 201. The smaller strut width and reduced twisting may provide lower strains, allows sheathing to smaller diameters and improvements in durability

There are various additional differences between prosthetic heart valves 100 and 200, including between the inner stents 105 and 205. For example, inner stent 105 includes commissure windows 155 in axial struts 151 that form part of the outflow end of the inner stent 105, whereas the commissure windows 255 of inner stent 205 extend beyond the main body of the remainder of the inner stent 205. This may allow the main body of inner stent 205 to be shorter than the main body of inner stent 105. In turn, the inner frame 205 may be able to extend a distance D4 beyond the outflow end of the outer stent 201 (see FIG. 2A) that is smaller than the distance D2 that the inner frame 105 extends beyond the outflow end of the outer stent 101 (see FIG. 1A). This may be desirable because, if there is less structure extending into the left (or right) ventricle, there is a smaller likelihood that the inner stent 205 will obstruct the left (or right) ventricular outflow tract, compared to inner stent 105. In other words, there is little or no structure between adjacent commissure windows 255 that would obstruct blood flow, while there is a relatively larger amount of stent structure between adjacent commissure windows 155. Additionally, during ventricular systole, there is relatively small amount of stent structure blocking blood from pressing against the outflow end of the prosthetic leaflets, meaning that the prosthetic leaflets may be faster to coapt with each other during ventricular systole as a result of the extension of the commissure windows 255 farther than adjacent stent structure. The shorter distance of the main body of the inner stent 205 may also provide for greater maneuverability of prosthetic heart valve 200 compared to prosthetic heart valve 100 during deployment and/or repositioning of the prosthetic heart valve. However, the design of inner stent 205 may result in the commissure windows 255 being more likely to deflect during use compared to commissure windows 155 of inner stent 105. In other words, commissure windows 255 may be more cantilevered than commissure windows 155. Thus, when the prosthetic valve assembly is under pressure, particularly when the prosthetic leaflets are closed and are resisting retrograde blood flow, the commissure windows 255 may have a greater tendency to deflect inwardly compared to commissure windows 155. To mitigate this possibility, the commissure windows 255 may include additional supports, in the form of support struts 257 described above, to form a “webbed commissure” structure. This “webbed commissure” structure may also provide a relatively atraumatic structure at the commissure windows 255, which may help avoid piercing any native tissue. Thus, the design of inner stent 205, including that of the commissure windows 255 and the support struts 257, allows for a relatively small protrusion of the inner stent 205 into the ventricle while also helping achieve optimal deflection of the commissure windows 255. These webbed commissures may also provide additional benefits to leaflet closure due to better fluid access to the free margin of the leaflets and a more robust sewing pattern at the commissures such that a metal retaining plate is not required and durability is improved.

Another difference between inner frame 105 and 205 is the position and structure of the v-shaped coupling members 154 (which include coupling aperture 112b) compared to coupling arms 212c (which include coupling aperture 212d). For example, v-shaped coupling members 154 are coupled to the main body of the inner frame 105 at two locations (the two struts that form the “v”-shape), whereas coupling arms 212c are coupled to the main body of inner frame 205 at only one location. This coupling at one location may be robust from a durability standpoint, while also avoiding twisting of struts during expansion and/or shape-setting. For relatively large sized prosthetic heart valves (which include relatively large frames), this may be especially important because the distance between the inner and outer frames may be relatively large. Thus, the ability to shape without twisting despite this relatively large distance may be desirable. Further, coupling arms 212c may be coupled to inner frame 205 so that aperture 212d extends beyond the inflow end of the inner frame 205 a distance (see FIG. 2A) that is smaller than the distance which aperture 112b extends beyond the inflow end of the inner frame 105 (see FIG. 1A). At least partially as a result of this, the inflow end of the outer frame 201 extends a distance D3 beyond the location of the coupling rivets 212e (see FIG. 2A) that is larger than the distance D1 which the inflow end of the outer frame 101 extends beyond the location of the coupling rivets 112c (see FIG. 1A). Release from the delivery system may be highly dependent on the angle of the pin or tab 222 relative to the axis and the distance D3. A central lumen that connects all the suture loops is pushed downward to release the suture loops. The angle from the pin or tab 222 to the interfering inner frame 205 may be important for release. The larger the angle, the easier the release. Thus, as can be best seen by comparing FIG. 2A to FIG. 1A, there is a relatively large amount of clearance in the atrial cells 211a around pin or tab 222 compared to the amount of clearance in the atrial cells 111a around pin or tab 122. As noted above, during deployment of the prosthetic heart valve 200, sutures or suture loops may loop around pins or tabs 222 to maintain a physical connection between the prosthetic heart valve and the delivery device. After deployment of the prosthetic heart valve 200, the suture loops may be advanced to slip the suture loops off the pins or tabs 222 to fully disconnect the prosthetic heart valve 200 from the delivery device. The larger amount of clearance around the pins or tabs 222, compared to the amount of clearance around pins or tabs 122, may make this process easier and reduce the likelihood of the sutures or suture loops failing to disconnected from the pins or tabs 222, for example via obstruction with other nearby stent structure.

For each of the frames 101, 105, 201, 205 described above, the wall thickness of each individual stent may be substantially constant, whether or not the inner frames 105, 205, have the same wall thicknesses as the corresponding outer frames 101, 201. However, in some embodiments, the stents that form the prosthetic heart valve 200 may have varying wall thickness. For example, the wall thickness of the outer stent 201 near the inflow end and/or near the outflow end may be reduced relative to the wall thickness of the remainder of the outer stent to reduce the stiffness of the atrial and/or ventricular tips of the outer stent 201 to reduce the likelihood of causing trauma to the tissue. For example, the wall thickness of the outer stent in the atrial cells 211a of the first row may be reduced compared to the remainder of the outer stent 201. In one example, only about half the atrial cells 211a in the first row, for example the inflow half, may have a reduced wall thickness compared to the remainder of the outer stent 201. The decrease in stent wall thickness may be gradual or abrupt, for example via a step change. The decreased thickness areas of the outer stent 201 may be created by any suitable method, including for example forming the outer stent 201 of a constant thickness, and then grinding down the stent to a smaller thickness at the desired locations. Additionally, or alternatively, the ventricular cells 211c in the first row may have a reduced wall thickness compared to the remainder of the outer stent 201. As with atrial cells 211a, the wall thickness of ventricular cells 211c may be reduced either gradually or abruptly, including at about half the length of the ventricular cells 211c on the outflow portion of those ventricular cells 211c. With this configuration, one or both tip ends of the outer stent 201 may be provided with reduced thickness to provide reduced stiffness relative to other portions of the outer stent 201 in order to reduce trauma to native tissue. It should further be noted that the atrial and ventricular tip ends of the outer stent 201 may be generally low strain locations compared to the other portions of the outer stent 201. As a result, reducing the stent wall thickness at these locations may not substantially hinder durability of the stent.

In addition or alternatively to reducing the stent wall thickness at one or both tip ends of the outer stent 201, the stent wall thickness near the central waist portion of the outer stent 201 may be increased relative to other portions of the outer stent 201 in order to increase stiffness in this area. For example, the second row of center cells 211f may have a stent wall thickness that is larger than immediately adjacent areas of the stent body 201. For example a portion of the center cells 211f, or the entire center cells 211f, which may include portions of adjacent cells 211b, 211e, 211g, may have a wall thickness greater than all remaining portions of the outer stent 201. When prosthetic heart valve 200 is implanted into a native valve annulus, as noted above, this center portion may be in contact with the native valve annulus while the atrial and ventricular ends wrap around the native valve annulus. As a result, when the heart contracts to pump blood, the center waist portion of the outer stent 201 may be subjected to a relatively large amount of contractile forces. While it may be desirable for the outer stent 201 to have some level of flexibility so as conform to the shape of the native valve annulus, it is typically not desirable for forces imparted on the outer stent 201 to transfer to the inner stent 205 and affect the prosthetic valve leaflets therein. Thus, by increasing stent wall thickness in the waist area of the outer stent 201, deformation of the outer stent 201 may be reduced during beating of the heart, which may help reduce any resulting deformation of the inner stent 205 and prosthetic leaflets positioned therein. The stent wall thickness of the waist portion of the outer stent 201 may be increased by any desired method. For example, the entire outer stent 201 may be formed with a constant thickness that is equal to the desired thickness of the waist portion, and the remaining areas of the outer stent 201 may be ground down to a smaller stent wall thickness. In other embodiments, the waist portion of outer stent 201 may be subsequently increased after the outer stent is formed having a constant stent wall thickness, for example by additive manufacturing, spray coating, dip coating, or any other suitable modalities.

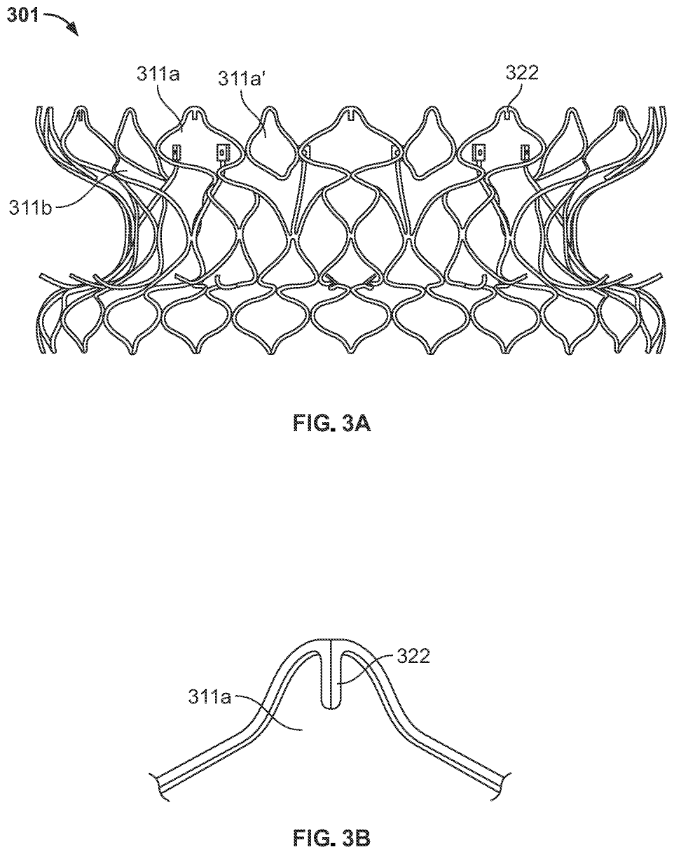

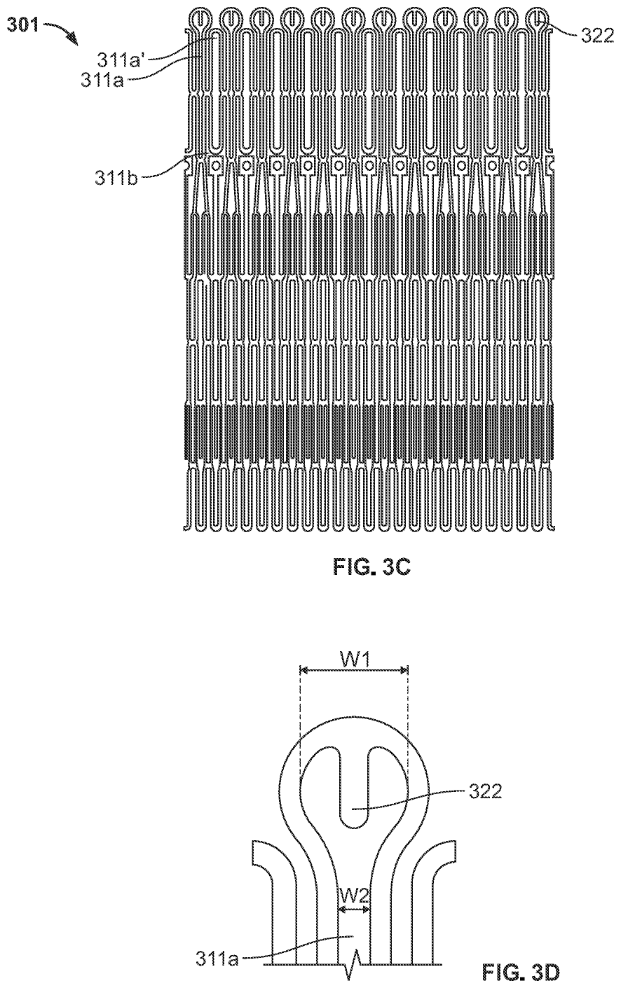

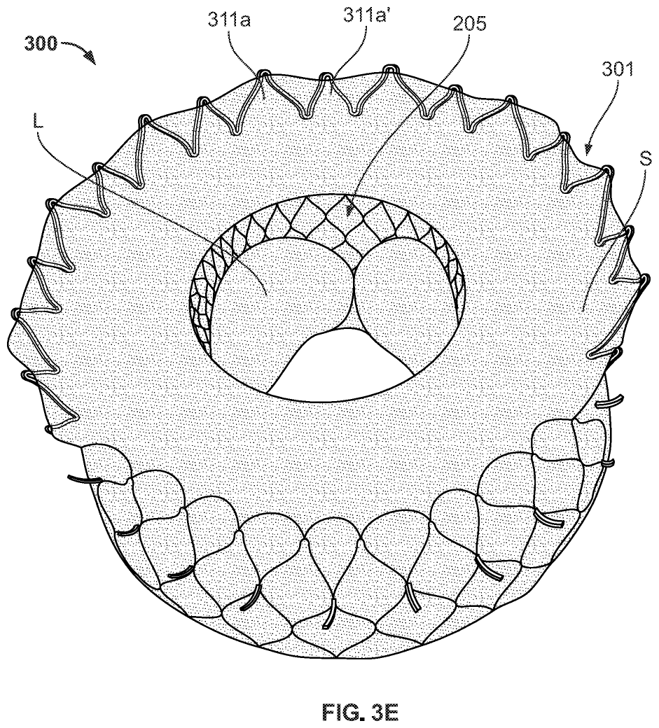

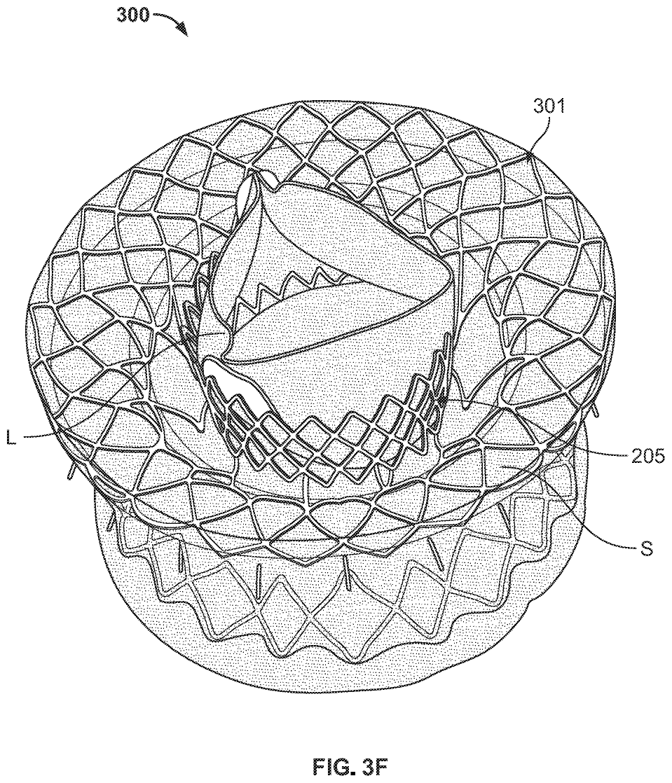

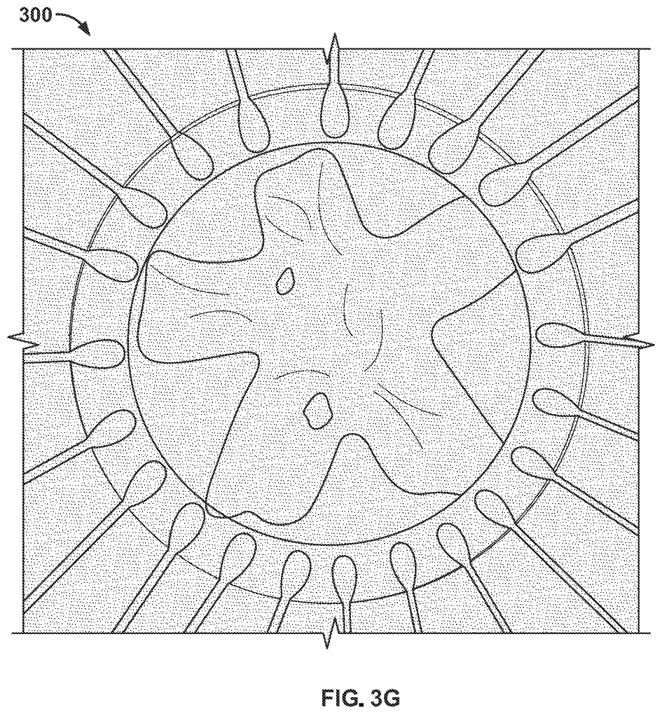

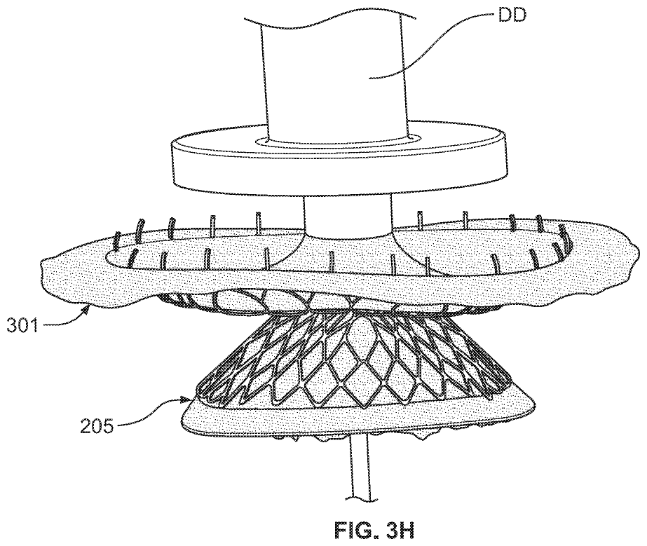

FIG. 3A illustrates an outer frame 301 of a prosthetic heart valve 300 in an expanded condition. Outer frame 301 may be part of a prosthetic heart valve 300 that is generally similar to prosthetic heart 200, and can be used with an inner frame that is substantially similar or identical to inner frame 205. Outer frame 301 may be identical to outer frame 201 in nearly all respects, other than the atrial tip portion, including the various alternatives (such as varying stent wall thicknesses) described in connection with outer frame 201.

Referring to FIGS. 3A and 3C, the inflow end of outer frame 301 may include a first row of atrial cells that include a first type of atrial cell 311a that alternates with a second type of atrial cell 311a′. The first type of atrial cells 311a and the second type of atrial cells 311a′ may both be general diamond shaped, with the first type of atrial cells 311a being slightly wider than the second type of atrial cells 311a′ in the circumferential direction. Outer frame 301 may include twelve of each type of atrial cell 311a, 311a′ for a total of twenty four cells. This is in comparison to outer stent 201, which may include twelve atrial cells 211a in the first row, with each atrial cell 211a being wider in the circumferential direction than either type of atrial cell 311a, 311a′. By increasing the number of atrial cells in outer stent 301, while maintaining the same or a similar diameter as outer frame 201, the atrial portion of outer frame 301 may be exposed to lower forces during loading, deployment, and while in use, which may reduce strain experienced by the atrial portion of the outer stent 301 and thus increase durability of the outer stent 301 compared to outer stent 201. Outer frame 301 may also include a second row of atrial cells 311b that are substantially similar to the second row of atrial cells 211b of outer frame 201. The main difference between atrial cells 311b and atrial cells 211b is that atrial cells 311b includes portions of the second type of atrial cell 311a′ extending into the atrial cell 311b. Thus, the bottom portions of atrial cells 211b, 311b may be substantially identical, including the coupling arms extending from the bottom apex of atrial cell 311b and toward the atrial or inflow end.

Still referring to FIG. 3A, it should be understood that pins or tabs 322 may be included with outer frame 301, much the same as pins or tabs 222 of outer frame 201. However, if twelve pins or tabs 322 are provided, they may be provided in the wider first type of atrial cell 311a, and omitted from the narrower second type of atrial cell 311a′. FIG. 3B is an enlarged view of a portion of one of the atrial cells 311a of the first type in an expanded condition, with pin or tab 322 shown. As can be seen in FIG. 3B, when the outer frame 301 is expanded, there is a relatively large amount of clearance around the pin or tab 322. With this configuration, after the outer frame 301 is fully expanded, it may be relatively easy to withdraw any suture loops that are positioned around the pin or tab 322, for example by advancing the suture loops forward and then beyond the free end of the pin or tab. When the outer frame 301 is in the collapsed condition, however, the clearance around the pin or tab 322 may significantly reduce (similar to the unexpanded condition shown in FIG. 3D). For example, the open space within atrial cell 311a adjacent the pin or tab 322 may be a first relatively large width W1 when the outer frame 301 is collapsed, but the remainder of the atrial cell may have an open space that is a relatively small width W2. By narrowing the open space W2 compared to open space W1, while the outer frame 301 is collapsed, any suture or suture loops surrounding the pin or tab 322 will be less likely to unintentionally detach, since the available space for the suture loops to move is reduced. However, once the outer frame 301 is expanded, as shown in FIG. 3B, there is a large amount of open space adjacent the pin or tab 322 to allow the suture loop to be disconnected to disconnect the outer frame 301 from the delivery device.

FIGS. 3E-3F are perspective views of prosthetic heart valve 300 in an expanded condition, with certain components omitted from FIGS. 3A-D included in FIGS. 3E-F. FIG. 3E shows prosthetic heart valve 300 from the atrial side, while FIG. 3F shows prosthetic heart valve 300 from the ventricular side. FIGS. 3E-F illustrate three prosthetic leaflets L coupled to the interior of the inner frame 205, with the prosthetic leaflets forming a valve assembly that is shown in FIGS. 3E-F in an open condition. Although three prosthetic leaflets L are shown, it should be understood that in other embodiments fewer or more than three prosthetic leaflets may be provided. Further, FIGS. 3E-F illustrate a skirt S, which may be formed of fabric, tissue, or combinations thereof, on the inner frame 205 and/or outer frame 301. The skirt S may be formed of a single piece of material or multiple pieces of material, and may extend over any one or more of the luminal and abluminal surfaces of the inner frame 205 and the outer frame 301.

Although prosthetic heart valve 300 may provide various beneficial features, there may be further room for improvement. FIG. 3G illustrates the interior of prosthetic heart valve 300 after having been transitioned into the collapsed condition, for example after having been collapsed into the sheath of a delivery device. As can be seen in FIG. 3G, the outer frame, inner frame, and/or the prosthetic leaflets positioned therein may infold and/or fold with a star-shape, which may be an undesired non-uniform collapse. As is described in more detail below, modification to the stent structure to prevent twisting at nodes between adjacent cells may assist with uniform folding during collapse.

FIG. 3H illustrates prosthetic heart valve 300 being deployed or expressed from the sheath of a delivery device DD. In FIG. 3H, most of the inner frame 205 has been deployed, with the atrial end of the inner frame 205 still held in the collapsed condition by the sheath of the delivery device DD. Further, the ventricular end of the outer frame 301, including barbs, have been deployed from the sheath of the delivery device DD in FIG. 3H, while the atrial portion of the outer frame 301 still remains constrained within the sheath of the delivery device DD. The particular expression (or shape which the prosthetic heart valve 300 takes during deployment) shown in FIG. 3H may be less than optimal. In this particular example, a phenomenon referred to as bursting is shown. For example, during expression or deployment of the prosthetic heart valve 300, it may be desirable for the ventricular tines or barbs of the outer frame to remain substantially vertical, with the ventricular anchor portion being substantially perpendicular to the axis of the prosthetic heart valve. This configuration may allow for easier positioning of the deployed ventricular portion of the valve prior to final engagement of the ventricular portion of the valve with the native tissue. It may also be desirable for the valve to protrude a relatively small distance into the ventricle to allow for easier repositioning within the annulus, if desired. Features that may assist with achieving these objectives are described in embodiments below.

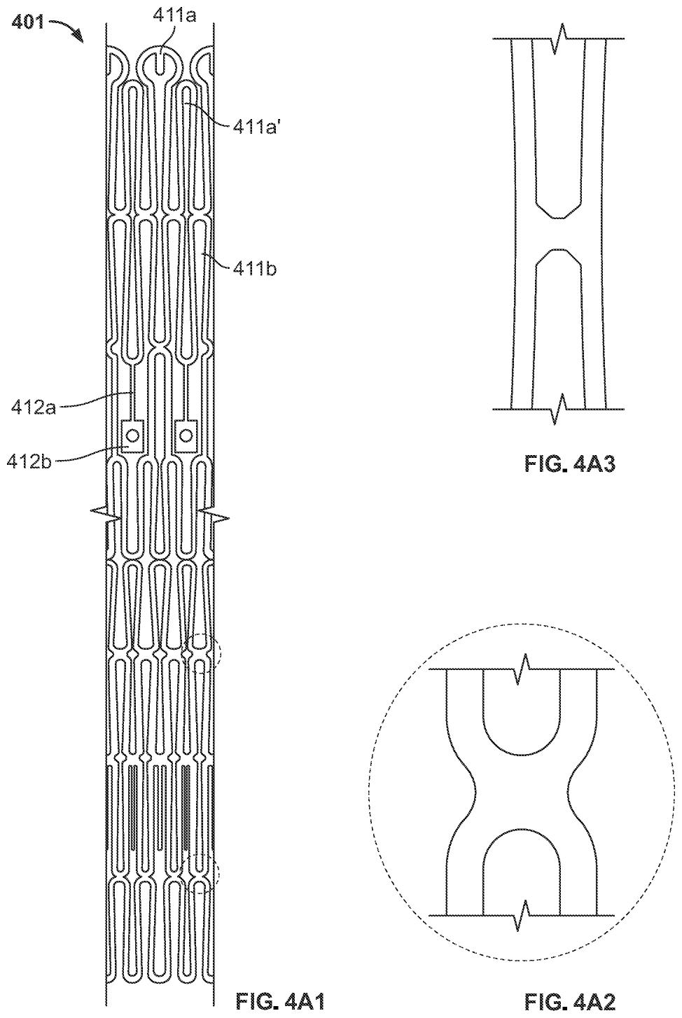

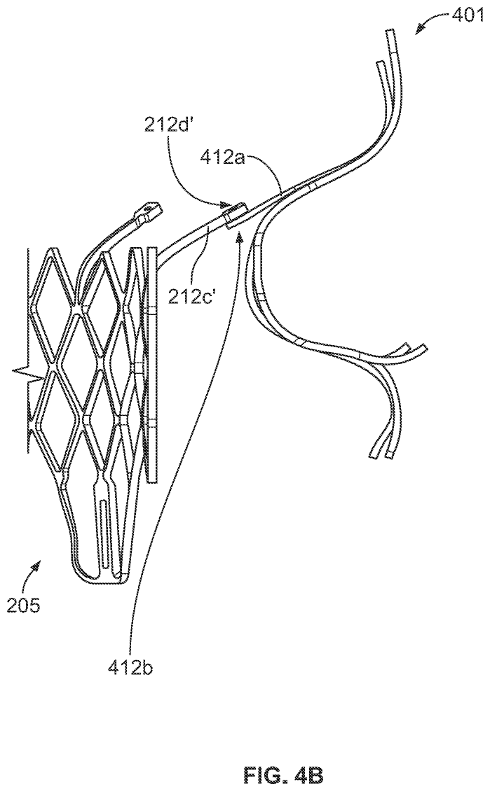

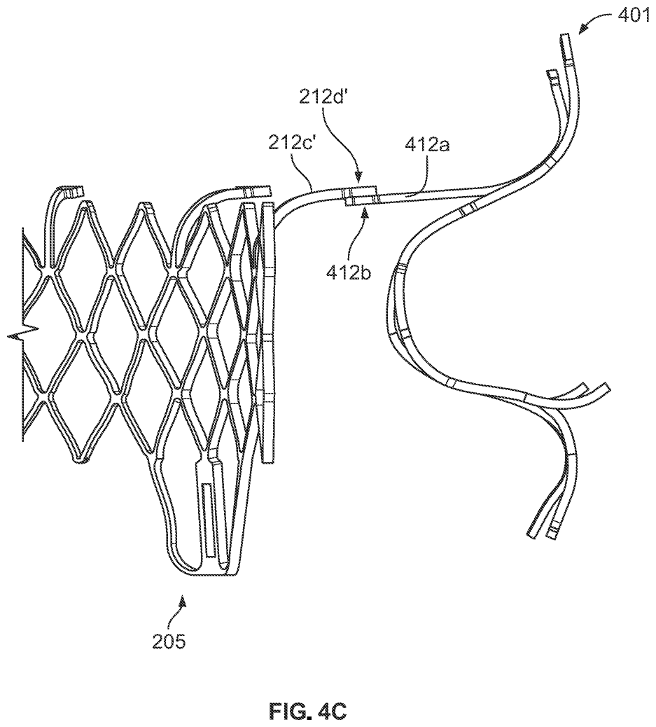

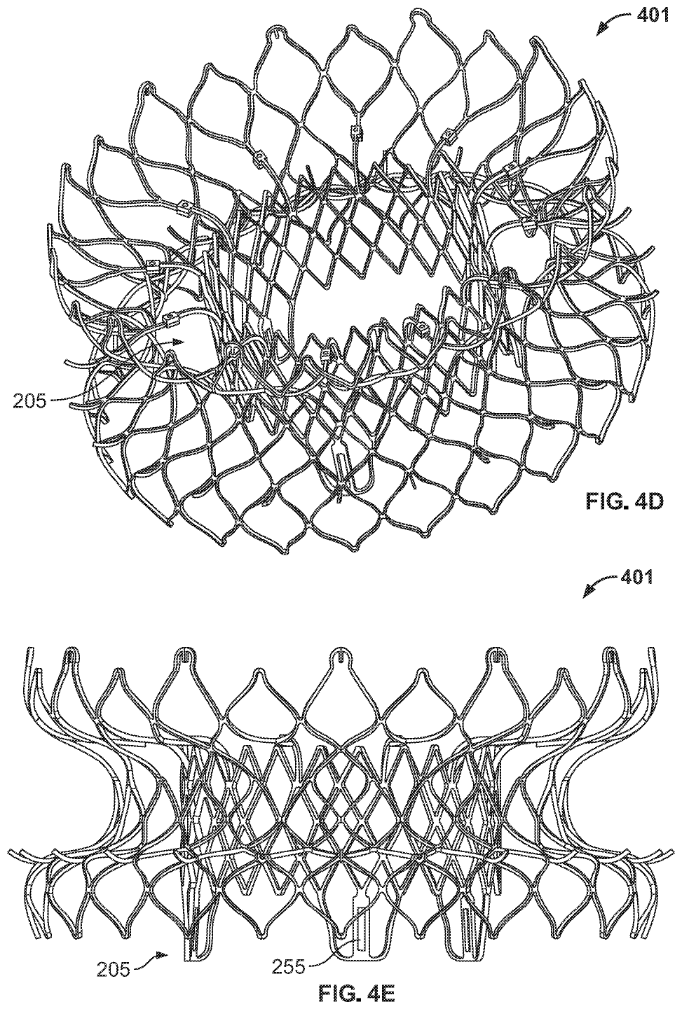

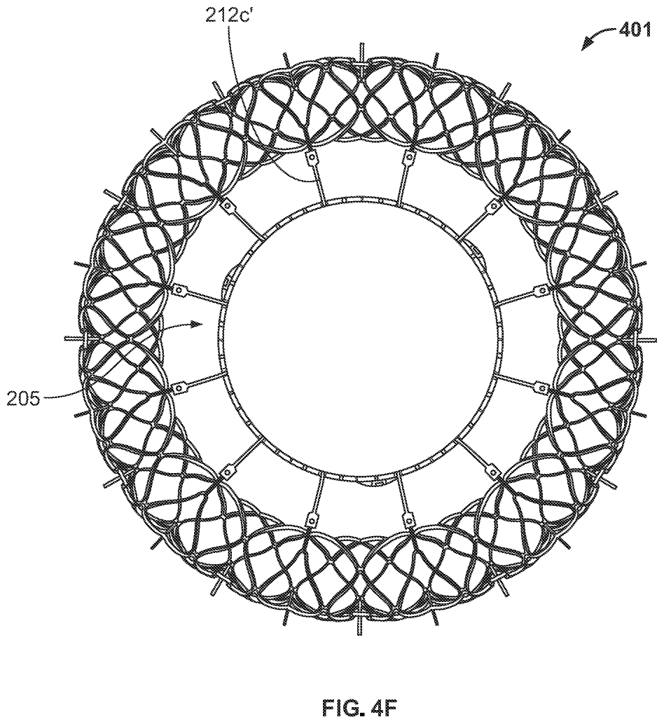

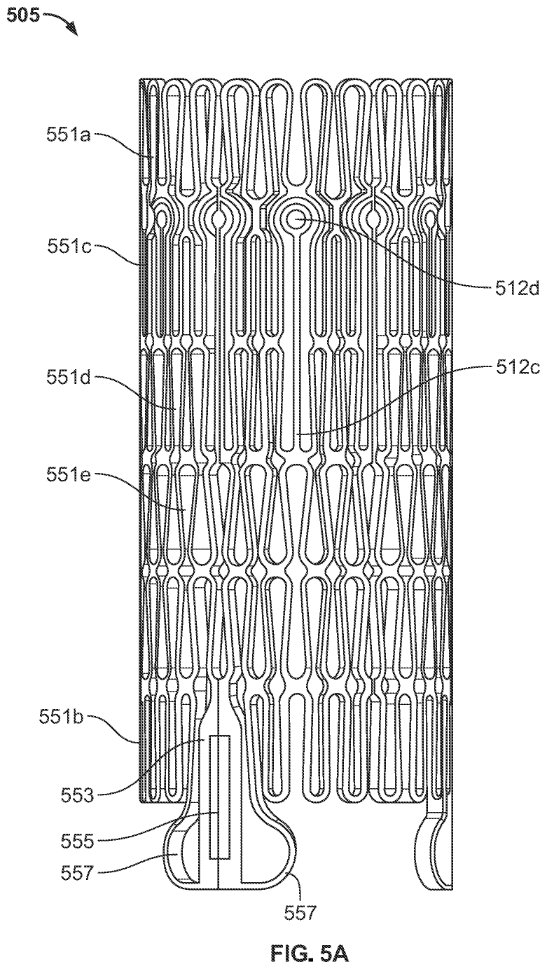

Additional modification may be made to the prosthetic heart valves 200, 300 and frames thereof in order to address at least some of the potential issued described above. FIG. 4A1 illustrates a portion of an outer frame 401 of a prosthetic heart valve, the portion of the outer frame being shown as if the outer frame was cut longitudinally and laid flat on a table. Outer frame 401 may be identical to outer frame 301 in most respects. For example, the ventricular portion of outer frame 401, which is near the bottom in the view of FIG. 4A1, may be similar or identical to the ventricular portions of outer frames 201 and 301. However, nodes between vertically adjacent cells may have indentations, contouring, or general “S”-shapes like shown in FIG. 4A2, compared to a more linear vertical connection between struts at the node, as shown in FIG. 4A3. The “S”-shaped strut connection at nodes may help to resist twisting of the stent at the nodes, particularly during collapse, which may in turn help achieve more uniform folding (including compared to the non-uniform folding shown in FIG. 3G). The atrial portion of outer frame 401, which is near the top in the view of FIG. 4A1, may be substantially identical to the atrial portion of outer frame 301, with the main exception being the position of coupling arms 412a. Whereas the coupling arms 212a of outer frames 201, 301 each extend from a bottom apex of the second row of atrial cells 211b, 311b, the coupling arm 412a of outer frame may extend in the opposite direction from a bottom apex of the second type of atrial cell 411a′. In other words, each second type of atrial 411a′, which is positioned between adjacent first types of atria cells 411a, may include a coupling arm 412a extending from a bottom apex of the second type of atrial cell 411a′ in a direction toward the ventricular or outflow end of the outer frame 401. Thus, compared to the coupling arms of outer frames 201, 301, the coupling arms 412a of outer frame 401 are connected to the outer frame more closely to the inflow end of the outer frame, but extend in the opposite direction. Thus, when in the collapsed condition, each coupling arm 412a and aperture 412b are positioned within one of the atrial cells 411b in the second row.

FIG. 4B is a cut-away side view of outer frame 401 coupled to inner frame 205 in an assembled and expanded condition. The inner frame 205 illustrated in FIG. 4B is identical to the inner frame of FIGS. 2D-E, with one difference. In particular, the shape of coupling arms 212c′ may be slightly different than coupling arms 212. For example, when expanded, the coupling arms 212c′ may have a slightly different flare or angle, so that the end of the coupling arms 212c′ that includes the aperture 212d′ is at an angle of between about 15 and 45 degrees relative to the central longitudinal axis of inner frame 205. The length of the coupling arms 212c′ may also be slightly greater than the length of coupling arms 212c. It should be clear that the reason for the difference in coupling arms 212c′ compared to coupling arms 212c is a result of the configuration of the coupling arms 412a of the outer frame 401. As can be seen in FIG. 4B, in the expanded condition of outer frame 401, the coupling arms 412a may extend at a similar angle as coupling arms 212c′ when the apertures 212d′, 412b of the coupling arms align. As with the other embodiments described herein, a rivet or other coupling structure may be used, for example by passing the coupling structure through both apertures 212d′, 412b, in order to couple the outer frame 401 to the inner frame 205.

With the configuration illustrated in FIG. 4B, the position and orientation of the coupling arms 212c′ relative to coupling arms 412a results in inner frame 205 being anchored to the outer frame 401 on the atrial side of the outer frame, for example rather than at or near the central waist of the outer frame. This is because the coupling arms 412a couple to the outer frame 401 on the atrial side of the outer frame 401, whereas coupling arms 212a couple to outer frame 201 near the central waist portion of outer frame 201. Further, this configuration may provide for more even collapsing of the prosthetic heart valve, reducing or eliminating the bursting effect shown in FIG. 3G. For example, as the prosthetic heart valve is drawn into a sheath of a delivery device to collapse the prosthetic heart valve in preparation of delivery, the coupling arms 212c′ and 412a will tend to collapse to a vertical alignment, generally parallel to the central longitudinal axis of the inner frame 205. As the loading or collapsing continues, the cylindrical portion of the inner frame 205 adjacent the ventricular portion of the outer frame 401 will act as an internal mandrel to guide the collapsing of the outer frame. In other words, the physical cylindrical structure of the inner frame 205 will tend to guide the ventricular portion of the outer frame 401 to collapse in a relatively symmetric and even fashion.

FIG. 4C illustrates a slightly different configuration of outer frame 401 and inner frame 205 compared to FIG. 4B. Although the structures shown in FIGS. 4B and 4C may be substantially similar or identical, the coupling arms 212c′ and 412a may be configured to have a slightly different orientation when in the expanded condition. Such a different configuration could be achieved, for example, by shape-setting the coupling arms 212c′ and 412a to have a slightly different angle in the unbiased or expanded condition. As shown in FIG. 4C, coupling arms 212c′ and 412a may be shape-set so that, when the inner frame 205 and outer frame 401 are assembled and expanded, the coupling arms may extend substantially orthogonal to the central longitudinal axis of the inner frame 205. This configuration may move the main body of outer frame 401 downwards or closer to the outflow end of the inner frame 205. By adjusting the angle of the coupling arms 212c′, 412a, the relative positions of the inner frame 205 and outer frame 401 may be altered. For example, the configuration illustrated in FIG. 4C may result in the inner frame 205 extending a smaller distance into the ventricle when implanted compared to the configuration illustrated in FIG. 4B, which may reduce the likelihood of obstructing the left or right ventricular outflow tracts. While FIG. 4C is a cut-away view, the entire assembled combination of outer frame 401 and inner frame 205, is shown in FIGS. 4D-F with the coupling arm configuration of FIG. 4C.

Inner frame 205 includes coupling arms 212c (or 212c′) that are fixed to the inner frame at the inflow or atrial side of the inner frame 205 where two adjacent cells 251a in the first row meet each other. This positioning of the coupling arms 212c (or 212c′) may result in a relatively large amount of movement of the inner frame 205 relative to the outer frame 401, particularly when the prosthetic valve assembly is in the closed condition and under pressure from a contracting ventricle. For example, referring to FIG. 4E, when the prosthetic heart valve is implanted, the prosthetic valve assembly is closed, and the prosthetic valve assembly is under pressure while the ventricle is contracting, upwards force is applied to the inner frame focused at or near the commissure windows 255. However, the main points of connection between the inner frame 205 and the outer frame 401 are via the coupling arms 212c′, which are on the opposite end of the inner frame compared to the commissure windows 255. Due to this relative spacing, the upward force from the contracting ventricle may result in relatively large amount of movement of the inner frame 205 with respect to the outer frame 401, the outer frame being clamped over the native valve annulus.

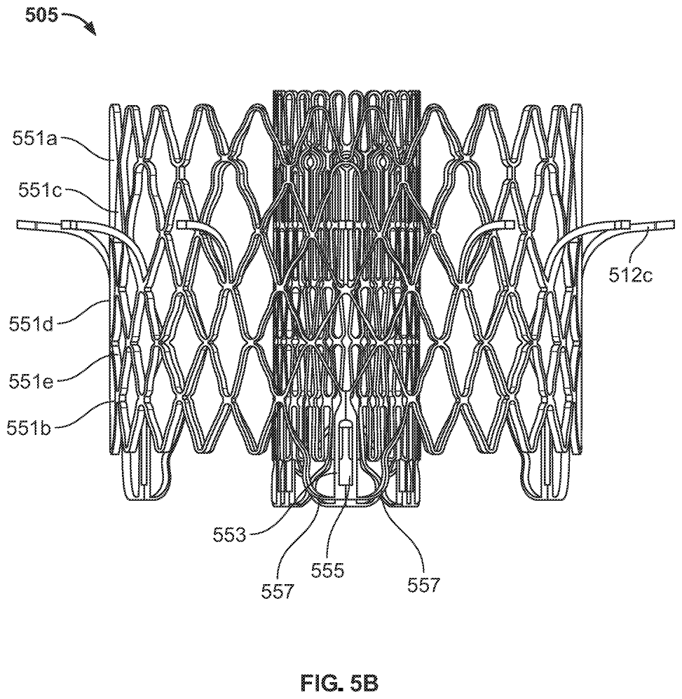

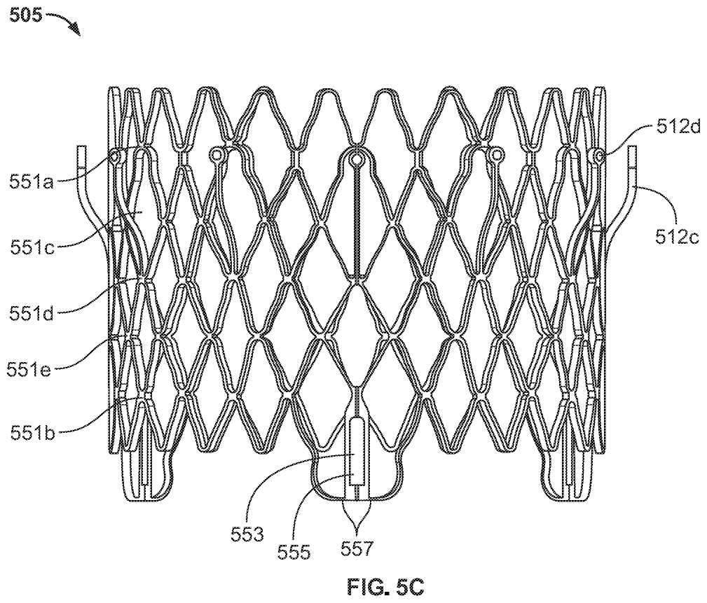

In order to reduce the potential movement of the inner frame relative to the outer frame when the prosthetic valve assembly is closed and the ventricle is contracting, the coupling arms may be provided in a different location on the inner frame. For example, FIG. 5A illustrates an inner frame 505 in a collapsed condition. Inner frame 505 may have a generally similar configuration as inner frame 205, but with certain differences.

In the illustrated example, inner frame 505 includes five rows of diamond-shaped cells, including a first row of cells 551a at the inflow end of the inner frame, and a second row of cells 551b at the outflow end of the inner frame. Third, fourth, and fifth rows of cells 551c-e may be positioned between the first and second rows. The inner frame 505 may include a plurality of commissure windows 555 formed in axial struts 553 that are generally similar to commissure windows 255. The axial struts 553 and commissure windows 555 may be generally aligned with and positioned between cells in the second outflow row 551b, although the commissure windows 555 and axial struts 553 may extend farther in the outflow direction than the cells in the second outflow row 551b. Additional support struts 557 may connect the axial struts 553 to the cells 551b, forming portions of the outflow row of cells. In addition to the additional rows of cells and slight configurational difference in commissure windows, the main difference of inner frame 505 compared to inner frame 205 is the position of coupling arms 512c. In particular, coupling arms 512c may have a first end coupled to the inner frame where two cells in the middle row 551d meet one another, the coupling arms 512c extending upwardly (toward the atrial or inflow end) and terminating in an aperture 512d. When inner frame is in the collapsed condition, the coupling arm 512c may extend substantially vertically parallel to the longitudinal axis of the inner frame 505, and be positioned within a cell in the third row of cells 551c. It should be understood that the cells in the third row of cells 551c may not all be identical to one another, may include a first type with a first shape to accommodate the coupling arms 512c, and a second type where the coupling arms 512c are omitted. Thus, in the illustrated example of inner frame 505, the coupling arms 512c are coupled to the inner frame 505 at or near the longitudinal center of the inner frame 505.

The coupling arms 512c of inner frame 505 may be shape-set into different configurations. In other words, when the inner frame 505 transitions from the collapsed condition shown in FIG. 5A to an expanded condition, the coupling arms 512c may transition to different shapes. Examples of different shapes are provided in FIGS. 5B-C, described in greater detail below.

FIG. 5B illustrates inner frame 505 after having transitioned to the expanded condition, with the coupling arms 512c shape-set into a first configuration. It should be understood that inner frame 505 is also shown in the collapsed condition in FIG. 5B for reference. As can be seen in FIG. 5B, the coupling arms 512c are shape-set to a configuration in which, when the inner frame 505 is expanded, the coupling arms 512c extend first upwardly (toward the atrium or inflow end) from the point of connection to the inner frame 505, and then radially outwardly so the terminal end portion of the coupling arm 512c (which includes aperture 512d) is substantially orthogonal to the center longitudinal axis of the inner frame 505. In other words, the coupling arms 512c shown in FIG. 5B are shape-set to have a similar shape in the expanded condition as coupling arms 212c′ shown in FIG. 4C. If inner frame 505 has this configuration, the outer frame may have a configuration similar or identical to outer frame 401 shown in FIG. 4C, and the inner and outer frames may be coupled together similarly, for example via rivets.

FIG. 5C illustrates inner frame 505 after having transitioned to the expanded condition, with the coupling arms 512c shape-set into a second configuration different than the first configuration of FIG. 5B. As can be seen in FIG. 5C, the coupling arms 512c are shape-set to a configuration in which, when the inner frame 505 is expanded, the coupling arms 512c extend first radially outwardly from the point of connection to the inner frame 505, and then upwardly (toward the inflow or atrium end) so the terminal end portion of the coupling arm 512c (which includes aperture 512d) is substantially parallel to the center longitudinal axis of the inner frame 505. In other words, the coupling arms 512c shown in FIG. 5B are shape set to have a similar shape in the expanded condition as coupling arms 212c shown in FIG. 2D. If inner frame 505 has this configuration, the outer frame may have a configuration similar or identical to outer frame 201 shown in FIG. 2B (or outer frame 301 in FIG. 3A), and the inner and outer frames may be coupled together similarly, for example via rivets.

By positioning the coupling arms 512c so that they coupled to the infer frame 505 about equidistantly between the inflow and outflow ends of the stent, when the prosthetic heart valve is being collapsed (for example when being loaded into the sheath of a delivery device), the atrial end of the inner frame 505 may avoid excessive collapse. In addition, when the prosthetic heart valve is implanted, the inner frame 505 may be positioned farther into the left atrium compared to prosthetic heart valves that incorporate inner frames having coupling arms similar to those shown in connection with inner frame 205. This may result in less protrusion of the inner frame 505 into the ventricle, which may help avoid obstruction of the LVOT (or RVOT if implanted in the tricuspid valve). Still further, the central positioning of the coupling arms 512c may help to maintain the entire inner frame 505 (or a large portion thereof) within the sheath of a delivery device during deployment of the outer frame. In other words, as the prosthetic heart valve is deployed from the sheath of a delivery device, the ventricular (or outflow) end of the outer frame may exit the sheath, including the waist portion of the outer frame, prior to most or any portion of the inner frame deploying from the sheath. This may be desirable because it may help avoid over-expansion of the ventricular or outflow end of the inner frame 505 during deployment, which may in turn avoid the likelihood of the tissue leaflets coupled to the inner frame being stretched or otherwise damaged during deployment.

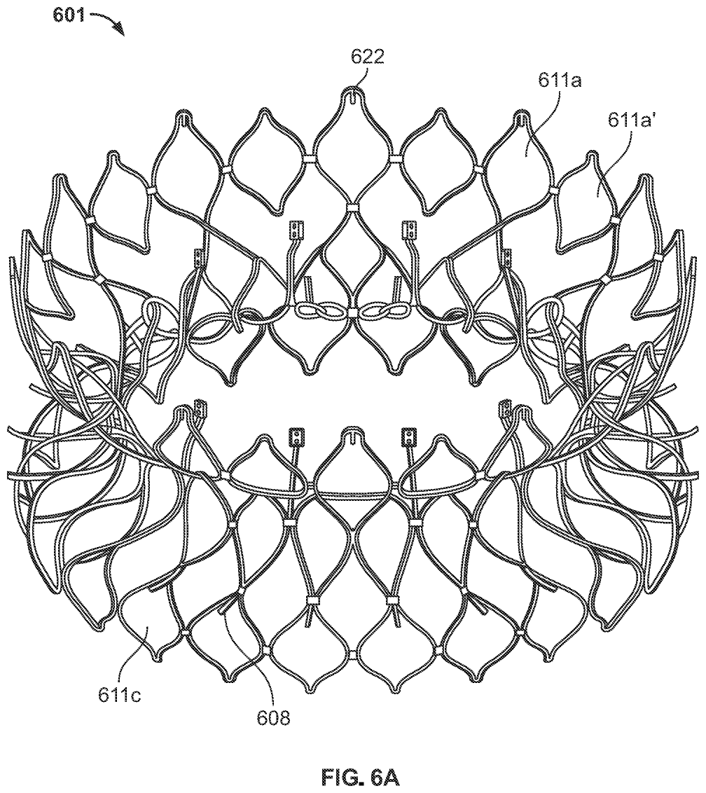

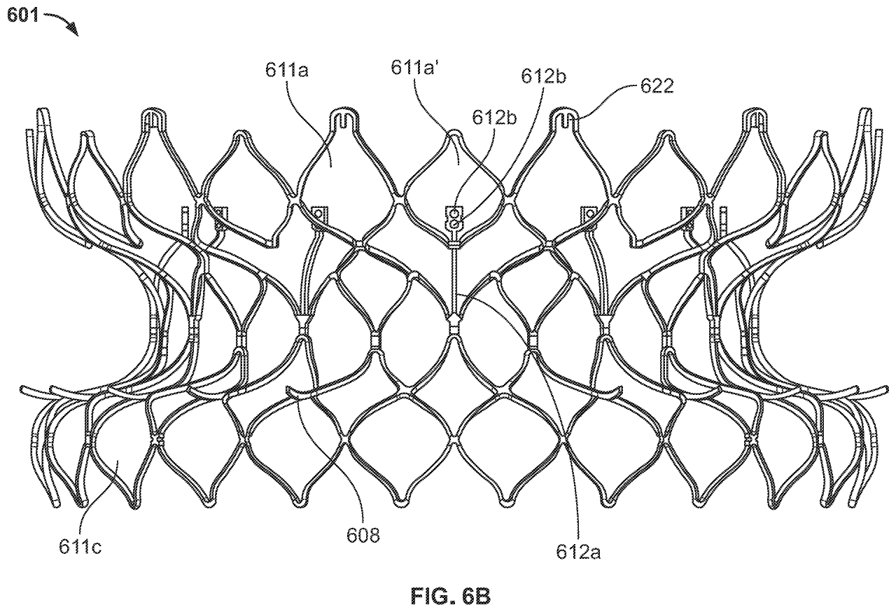

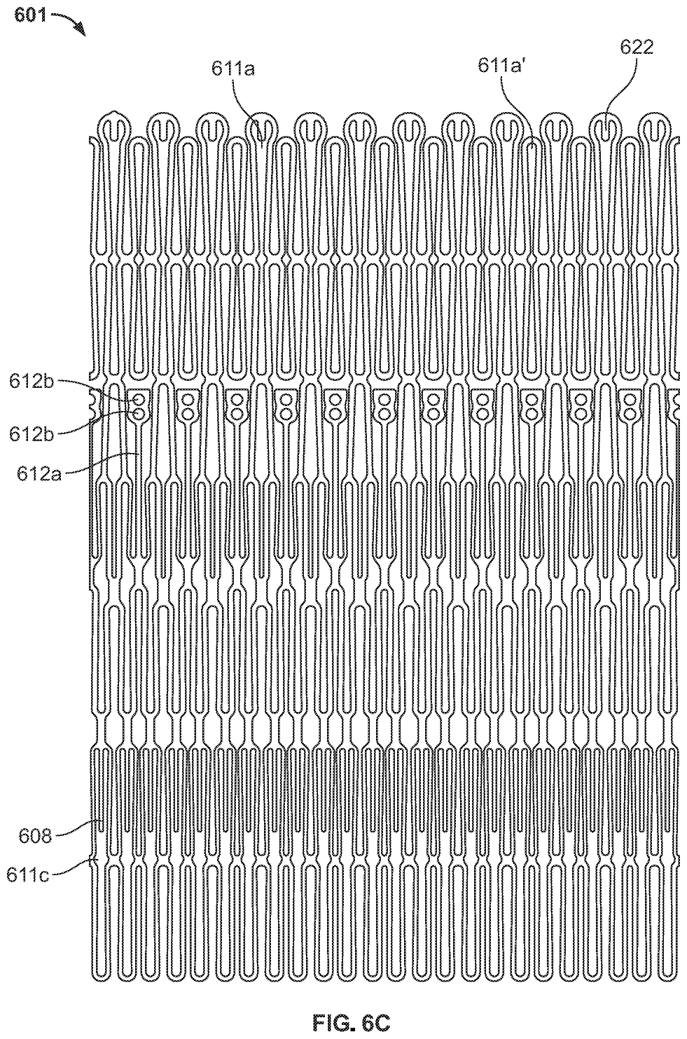

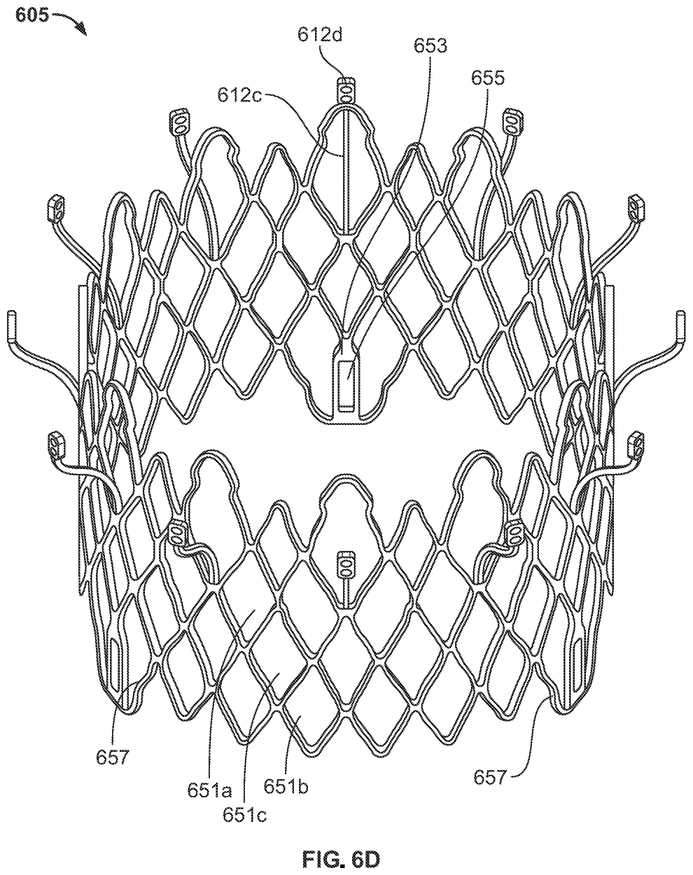

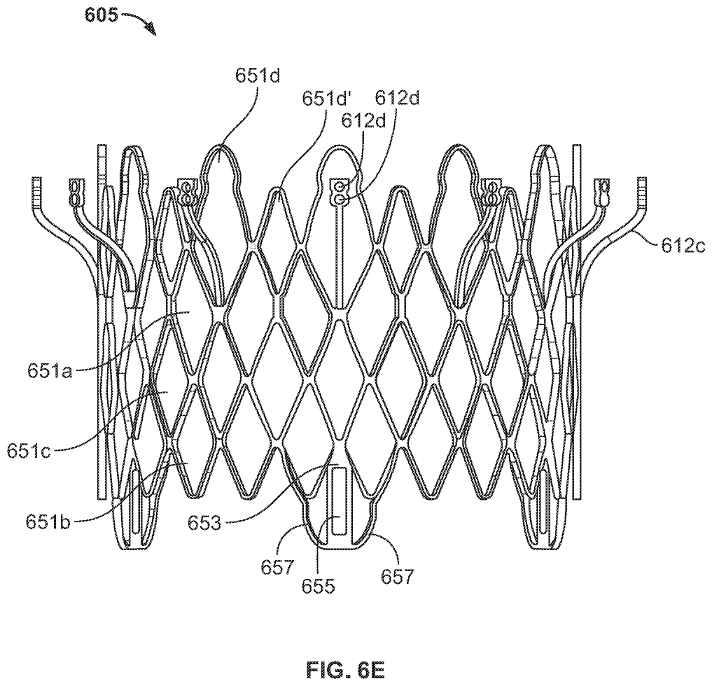

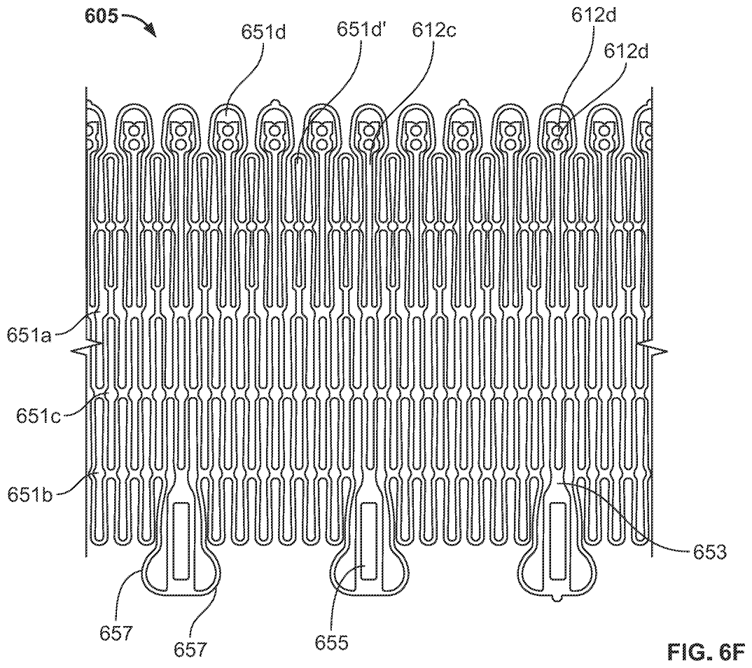

FIGS. 6A-B illustrate an outer frame 601 of a prosthetic heart valve in an expanded condition. Outer frame 601 may be part of a prosthetic heart valve that is generally similar to prosthetic heart 200. Outer frame 601 may be similar or identical in many respects to outer frames 201 and 301, with certain exceptions. Thus, features not explicitly described in connection with outer frame 601 should be understood to be similar or identical to the corresponding features of outer frames 201 and 301.

Referring to FIGS. 6A-B, outer frame 601 may include a plurality of rows of diamond shaped cells that are generally similar to those of outer frames 201 and/or 301. For example, the outer frame 601 may include twenty-four diamond-shaped cells in the ventricular-most rows of cells 611c, which may be referred to as the “ventricular petals” as they are intended for positioning on a ventricular side of the native mitral valve annulus. Whereas outer frames 201 and 301 include two ventricular rows of cells (e.g. cells 211c, 211d), outer frame 601 includes only a single row of ventricular cells 611c. As a result, outer frame 601 may contain six rows of cells, compared to outer frames 201, 301 which include seven rows of cells. Further, outer frame 601 may include three rows of diamond-shaped cells at a central waist portion of the outer frame, with these three rows being substantially similar or identical to the first, second, and third rows of center cells 211e-g of outer frame 201. Toward the atrial side, the outer frame 601 may include two rows of atrial cells. The second row of atrial cells (positioned adjacent the inflow-most row of cells) may be similar or identical to the second row of atrial cells 311b of outer frame 301, which are generally diamond-shaped but for the interruption from the first row of atrial cells (and in particular the second type of atrial cells 611a′). The first row of atrial cells are positioned at the inflow-most end of the outer frame 601, and may be referred to as atrial petals because they are intended for positioning on the atrial side of the native mitral valve annulus. Generally similar to outer frame 301, the first row of atrial cells of outer frame 601 may include a first type of atrial cell 611 alternating with a second type of atrial cell 611a′. The first type of atrial cell 611a may extend slightly farther in the inflow direction than the second type of atrial cell 611a′, although both are generally diamond-shaped. The first type of atrial cell 611a may include an atrial pin or tab 622, similar to the corresponding atrial pins 322. The first row of atrial cells of outer frames 601 and 301 may be identical in most respects. However, one difference is that the first type of atrial cells 611a extend slightly farther in the inflow direction, whereas the atrial petal tips of the first row of atrial cells in outer frame 301 are all at substantially the same height. Another difference is that the atrial petal tips of the first type of atrial cells 611a, where the atrial pin or tab 622 resides, may be slightly narrower in outer frame 601 compared to outer frame 301. This latter difference may be only recognizable in the deployed or relaxed state of the outer frame 601. In other words, in the unexpanded condition of the outer frame 601 shown in FIG. 6C, the first and second types of atrial cells 611a, 611a′ are very similar or identical to the corresponding first and second types of atrial cells 311a, 311a′ of outer frame 301 when in the unexpanded condition, shown in FIG. 3C. Atrial cells 611a and 611a′ may be designed such that atrial cell 611a with the atrial pin or tab 622 collapses immediately upon sheathing to capture the delivery system sutures securely, while atrial cells 611a′ collapse subsequently and the free ends curve radially inward and out of the way of atrial cells 611a providing a means for the use of smaller delivery systems equal to about 12 times the width of atrial cell 611a.

Other than the arrangement of the rows of cells, outer frame 601 may have two additional notable differences compared to outer frames 201, 301. First, the ventricular tines or barbs 608 of outer frame 601 may extend from an apex of each cell 611c in the ventricular row of cells (the apex being positioned opposite the outflow-most portion of each cell 611c). Thus, outer frame 601 may include twenty-four ventricular tines or barbs 608, with each ventricular tine or barb being equidistantly spaced from circumferentially adjacent ventricular tines or barbs. This configuration is more similar to outer frame 101 shown in FIG. 1B. Second, outer frame 601 may include coupling arms that have a slightly different configuration than those of outer frame 201. For example, while the coupling arm 612a itself may be similar or identical to coupling arms 212a of outer frame 201, the free end portion of coupling arm 612a may include two vertically spaced apertures 612b, whereas coupling arms 212a include only a single aperture 212b. Otherwise, the coupling arms 612b may be similarly coupled to outer frame 610 where two adjacent center-most cells at the waist meet one another, with coupling arms 612a being positioned at every other meeting of adjacent center-most cells, so that a total of twelve coupling arms 612a are provided along the row of twenty-four center-most cells. The function of, and various possible alternative characteristics of, coupling arms 612a is described in more detail below.

FIGS. 6D-E illustrate an inner frame 605 of a prosthetic heart valve in an expanded condition. Inner frame 605 may be part of a prosthetic heart valve that is generally similar to prosthetic heart 200. Inner frame 605 may be particularly suited for use with outer frame 601. Inner frame 605 may be similar or identical in many respects to inner frame 205, with certain exceptions. Thus, features not explicitly described in connection with inner frame 605 should be understood to be similar or identical to the corresponding features of inner frame 205. FIG. 6F illustrates the inner frame 605 in an unexpanded condition, and as if cut longitudinally and laid out on a table.

Referring to FIGS. 6D-F, inner frame 605 may be primarily intended to support prosthetic leaflets, and may designed to foreshorten axially upon radial expansion. For example, inner frame 605 may include five rows of cells, most or all of which are generally diamond-shaped. In particular, inner frame 605 may include first, second, and third rows of diamond-shaped cells 651a-651c that are generally similar or identical to rows 251a-c of inner frame 205. The three rows of cells 651a-651c may each include twenty-four cells, although some of the cells in the second row 651b may be formed partially with the webbed commissures, described below. The third row of cells 651c may be positioned between the first row of cells 651a and the second row of cells 651b. The main differences between inner frame 605 and inner frame 205 relate to the commissure windows 655, and an additional row of cells on the inflow-most end of the inner frame 605, each described below.

Inner frame 605 may have a generally similar configuration of webbed commissure windows 655 formed in axial struts 653, and supported by two additional support struts 657 each for the same purpose as described above in connection with inner frame 205. However, the axial struts 653 forming commissure windows 655 may extend from a location where two adjacent cells 651b in the same row meet. In other words, each axial strut 653 extends from an outflow apex of a cell 651c in the third row of cells. This is in contrast to axial struts 253 of inner frame 205 that extend from outflow apices of cells in the second row 251b. This change results in the webbed commissures of inner frame 605 only extending beyond the second row of cells 651b in the outflow direction a length of about one half the axial length of a cell 651b in the second row. The webbed commissures of inner frame 605, compared to those of inner frame 205, may allow for about the same height for connection of the prosthetic leaflets to the webbed commissures, while reducing the distance which the inner frame 605 extends into the left ventricle upon implantation, which may reduce the likelihood (or amount of) obstruction of the LVOT (or RVOT if implanted in the tricuspid valve position). The additional support struts 657 may be generally similar to support struts 257 of inner frame 205, with one main difference being the support struts 657 connect the axial strut 653 to the inner frame 605 at side apices of cells 651b in the second row (as opposed to the inflow or outflow apices). The support struts 257 of inner frame 205, on the other hand, couple to outflow apices of cells 251b in the second row. Similar to the webbed commissures of inner frame 205, the positioning of the webbed commissures of inner frame 605 may allow the free edge of the prosthetic leaflets to be fully exposed to blood flow without the inner frame blocking the flow, which may provide improved leaflet closing dynamics (e.g. faster leaflet coaptation) and may reduce the likelihood or possibility of leaflet wear due to contact with the inner frame 605. Also, as with inner frame 205, the webbed commissures of inner frame 605 may be structurally stable at least in part due to the support struts 657 that create the “web” of the webbed commissure. Another benefit is ease of manufacture/assembly of the leaflet and a more robust and durable commissure.

Referring to FIG. 6F, it may be desirable to allow for the axial struts 653 to deflect radially inwardly during normal operation of the prosthetic heart valve that incorporates inner frame 605. For example, adjacent prosthetic leaflets are coupled to each other and to the inner frame 605 via commissure window 655. Thus, during ventricular systole when the prosthetic leaflets coapt and are subjected to the relatively large pressures of ventricular systole, the force on the prosthetic leaflets is transmitted to their connection to the inner frame 605, including at axial struts 653. If the axial struts 653 with commissure windows 655 were extremely rigid, a relatively high amount of stress would be applied to the prosthetic leaflets where they couple to the axial struts 653. However, by designing the axial struts 653 to be capable of deflecting inwardly, some amount of the stress that would otherwise be placed on the prosthetic leaflets is instead distributed to the inner frame 605. By reducing the stress on the prosthetic leaflets, the prosthetic leaflets may be more durable than if the prosthetic leaflets had to bear a relatively higher amount of the stress during ventricular systole. The webbed commissures of inner frame 605 may be specifically designed so that they are capable of flexing between about 0.5 mm and about 1.5 mm, including about 1.0 mm, radially inwardly (measured at the tips of the axial struts 653) during ventricular systole. Some of the parameters that that may be leveraged to fine-tune the amount of deflection that will occur in the axial struts 653 include (i) the wall thickness of the stent (in the direction into the page in the view of FIG. 6F); (ii) the width of the node (in the left-to-right direction in the view of FIG. 6F) where the axial strut 653 connects to the outflow apex of the adjacent cell in row 651c; and (iii) the axial position along axial strut 653 where supporting struts 657 connect to axial strut 653. In one embodiment, to achieve a desired amount of deflection, the width of the node may be larger than the wall thickness of the inner frame 205. For example, the ratio of the width of the node to the wall thickness may be greater than 1 and less than 2, including about 1.3, about 1.4, or about 1.5. It may be preferably, as shown in FIG. 6F, to couple the support struts 657 to the terminal (otherwise free) end of the axial strut 653. This may reduce deflection to help achieved the desired amount of deflection. Further, by connecting the support struts 657 to the outflow-most end of the axial struts 653, the support struts 657 may curve outwardly and back inwardly at positions beyond the ends of the cells in the second row of cells 651b. This curvature may provide for extra length of the support struts 657. This extra length may assist in ensuring that the cells connected to the opposite ends of the support struts 657 are capable of expanding as intended. In other words, when the inner frame 205 transitions to the expanded condition shown in FIGS. 6D-E, the axial struts 653 do not undergo any conformational change, while the adjacent cells do. The extra length of support struts 657 resulting from the above-described contouring provides for cells adjacent the axial struts 653 that are not restricted from expanding, despite the axial struts themselves remaining unchanged during expansion. The features described in this paragraph may apply substantially similar to the other webbed commissures described in other embodiments herein.

In some embodiments, the wall thickness of the inner frame 605 may be consistent throughout the inner frame 605. However, in some embodiments, a step change in reduced (or increased) wall thickness may be provided to further fine tune the amount of deflection experienced by the axial struts 653. If a step change is made in wall thickness, it may be desirable for that change to occur at a point in the inner frame 605 where there are relatively little forces experienced, which may minimize the likelihood of problems occurring due to forces acting at the step change. Such a desired location may include, for example, near the middle of a strut between connections to other struts or cells. For example, referring to FIG. 6F, the wall thickness of the inner frame 605 may be relatively small between the terminal end of axial struts 653, and either (i) an axial mid-point along the bottom “V”-shape of the second row of cells 651b or (ii) an axial mid-point along the top “V”-shape of the second row of cells 651b. Such a step change in wall thickness may allow for the majority of the inner frame 605 to have the thickness desired for structural stability, while reducing (or increasing) the wall thickness at and around the commissure webs to allow for greater deflection during ventricular systole.

Inner frame 605 may also include a fourth row of cells at the inflow-most (or atrial) end of the frame, whereas inner frame 205 has no corresponding row of cells at that position. The fourth row of cells may include a first type of atrial cell 651d that alternates with a second type of atrial cell 651d′ for a total of twenty-four cells in the atrial-most row. The second type of atrial cell 651d′ may be a diamond shaped cell coupled to a runner between pairs of cells 651a in the first row. The first type of atrial cell 651d may be generally diamond-shaped, but larger than the second type of atrial cell 651d′. The first type of atrial cell 651d may extend a greater length in the inflow direction than the second type of atrial cell 651d′, and the first type of atrial cell 651d may surround corresponding coupling arms 612c, described in greater detail below, at least when the inner frame is in the unexpanded condition shown in FIG. 6F. Although inner frame 605 is described as having four rows of cells, it may also be appropriate to describe inner frame 605 as including five row of cells of the webbed commissures are counted as a row of cells separate from the second row of cells 651b.

Similar to inner frame 205, inner frame 605 may include coupling arms 612c coupled to points where every other pair of adjacent cells 651a in the first row meet, resulting in a total of twelve coupling arms 612c. The main difference between coupling arms 612c and 212c is that coupling arms 612c include two vertically separated apertures 612d, whereas coupling arms 212c only include a single aperture 212d.

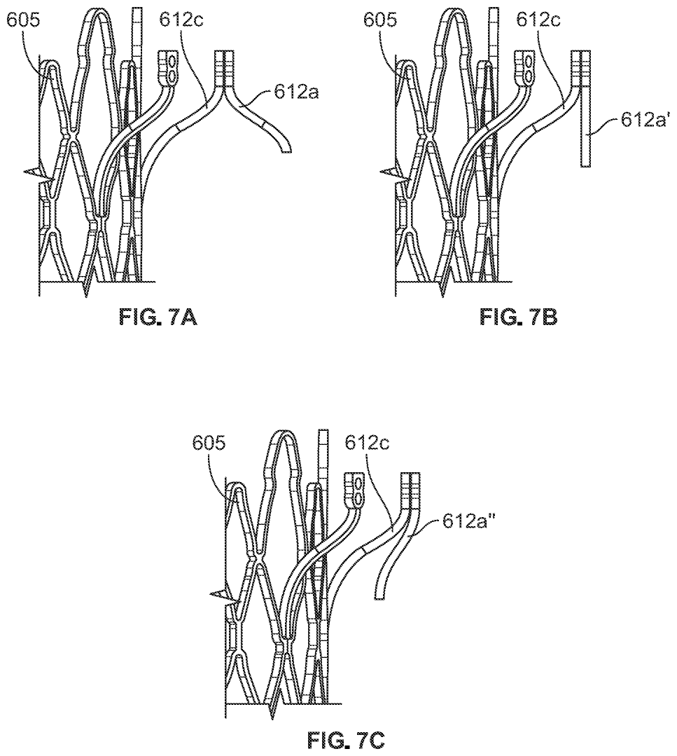

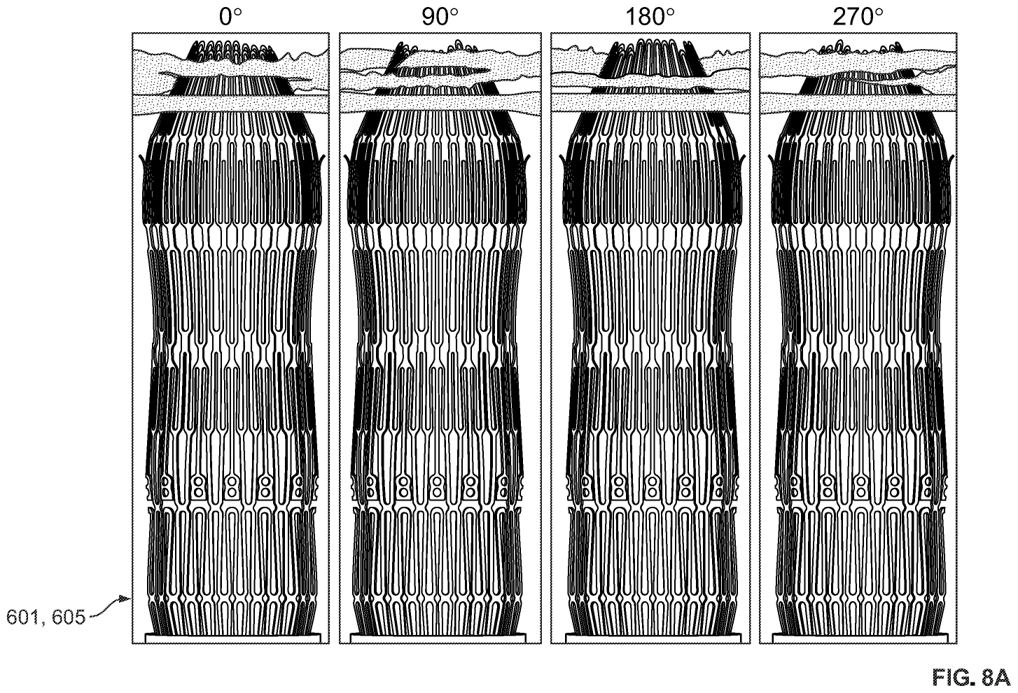

As should be understood from the above description, the outer frame 601 may be attached to the inner frame 605, and additional components such as prosthetic leaflets and fabric cuffs/skirts may be attached to the frame(s) in order to form a self-expanding prosthetic mitral (or tricuspid) valve. In use, a single size inner frame 605 may be used with different sized outer frames 601 in order to accommodate different patient anatomies. For example, the inner frame 605 may have a 27 mm diameter, and the outer frame 601 may come in different diameters (at the central waist) of 32 mm, 36 mm, or 40 mm. It should be understood that these are merely exemplary sizes, and the inner frame could be provided in additional sizes, and the outer frame could be provided in more or fewer sizes than listed above. The size of the outer frame may affect the particular geometry of the coupling arm 612a, particularly if only a single size of inner frame 605 is provided. For example, the outer frame 601 illustrated in FIGS. 6A-C may have a diameter of 40 mm at the central waist. The coupling arm 612a, when in the expanded condition, extends upward (or in the atrial direction) from its point of connection to the outer frame 601, and then radially inwardly. As shown in FIG. 7A, when the inner frame 605 is assembled to the outer frame 601 (having a 40 mm diameter), the coupling arms 612a, 612c extend toward each other, curving near their free ends so that the terminal flat plates press against each other in a vertical direction and the two apertures 612b align with the two apertures 612d. The terminal free ends (or flat plates) of the coupling arms 612a, 612c may be coupled via suturing through the corresponding apertures 612b, 612d, or by any other suitable fastener (such as rivets). The geometry of the coupling arms 612a, 612c may result in the arms meeting at a point about mid-way between the outer diameter of the inner frame 605 and the inner diameter of the outer frame 601. Although not shown, a buffer material (such as fabric or tissue) may be provided between the terminal free ends of the coupling arms 612a, 612c, for example to prevent direct metal-to-metal contact. Although fasteners such as rivets may be used to couple any of the inner and outer frames described herein, it may be preferable to use sutures to fasten the coupling arms for ease of manufacturing (e.g. the inner and outer frames may be coupled to one another relatively late in the manufacturing process). In addition, although rivets are a suitable fastening option, rivets may create a potential for galvanic corrosion that may occur when a metal rivet is used to couple metal frames, where the metal of the rivet is dissimilar to the metal of the frames. The use of suture fasteners may eliminate this potential issue.

If outer frame 601 has a diameter smaller than the 40 mm diameter shown in FIGS. 6A-C, the coupling arm of the outer frame may have a slightly different geometry. For example, if outer frame 601 has a 36 mm diameter at the central waist, the coupling arm 612a′ may extend substantially fully vertically from the outer frame, as shown in FIG. 7B. Because the inner frame 605 in FIGS. 7A-B is identical, the different geometry of coupling arm 612a′ of the smaller diameter outer frame 601 may be necessary in view of the smaller diameter of the outer frame 601. This same concept may be applied to a variety of sizes of outer frame 601. For example, FIG. 7C illustrates an even smaller size outer frame 601, which may have a 32 mm diameter at the central waist, while again inner frame 605 remains identical. In order to accommodate this even smaller size outer frame 601, coupling arms 612a″ may extend vertically from its connection point with the outer frame 601, and then radially outwardly, before returning to a vertical direction. Additional diameters of outer frame 601 may be provided with inner frame 605, and the coupling arms 612a of the outer frame may be modified in ways other than those explicitly illustrated to allow for coaxial positioning of the outer frame around the inner frame, with uniform contact between coupling arms 612a and 612c. It should be understood that in FIGS. 7A-7C, only a single coupling arm of the outer frame is illustrated, with the remainder of the outer frame omitted, for clarity of illustration.

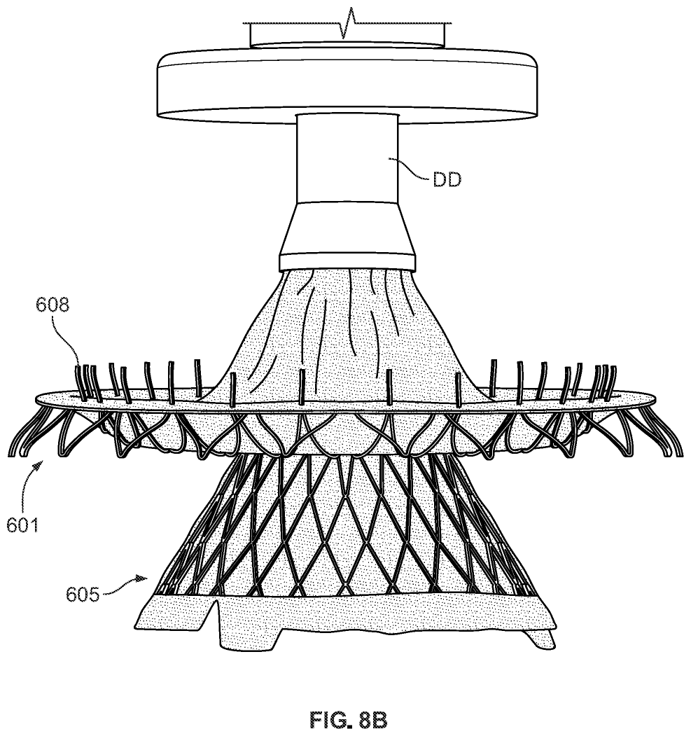

Referring back to FIG. 6B, whether outer frame 601 has a relatively large diameter or a relatively small diameter, the coupling arms 612a preferably couple to the outer frame 601 at or near the middle of the central waist portion of the outer frame, for example at or near the smallest diameter portion of the outer frame. Both coupling arms 612a of the outer frame 601 and coupling arms 612c of the inner frame 605 are preferably balanced in stiffness relative to each other so that, as the inner and outer frame assembly collapses or expands, the point where the coupling arms couple remains generally between the inner and outer frames, without significantly moving away from the outer frame toward the inner frame, or significantly moving away from the inner frame toward the outer frame. This may help maintain balance between the inner and outer frames, while avoiding excess strain being placed on coupling arms of the inner frame or the outer frame during collapsing or expansion. The positioning and design of the coupling arms may provide additional benefits as well. During a typical transseptal delivery, the ventricular end of the prosthetic heart valve is deployed from the delivery device first, with the atrial end deploying last. Because the coupling arms 612c of the inner frame 605 are positioned on the atrial side of the inner frame, with the actual point of connection of the coupling arms 612c to coupling arms 612a being near the atrial-most end of the inner frame, the inner frame 605 will tend to be positioned a significant distance within the delivery sheath, even as the ventricular end of the outer frame 601 begins to deploy from the delivery sheath. By maintaining a significant length of the inner frame 605 collapsed within the delivery device while the ventricular end of the outer frame 601 begins to deploy, the prosthetic heart valve may deploy in a more controlled manner Δn example of this preferred mid-deployment configuration is shown in FIG. 8B. For example, as shown in FIG. 8B, during deployment, the tines or barbs 608 are substantially vertically oriented, and the ventricular portion of the outer frame 601 is substantially perpendicular to a longitudinal axis of the prosthetic heart valve. In other words, if the inner frame 605 began to deploy at the same time the ventricular end of the outer frame 601 began to deploy, it may become more difficult to obtain desired positioning of the ventricular end of the outer frame 601 relative to the ventricular side of the valve annulus prior to continuing deployment of the prosthetic heart valve.

It should be understood that many of the benefits described above in connection with inner frame 205 and outer frames 201, 301 may similarly apply to inner frame 605 and outer frame 601, and thus these benefits are not all repeated herein. However, certain benefits are described or reiterated. Outer frame 601 may have a relatively large number of cells, in particular compared to the prior art outer frame 101. This additional structure may provide a more uniform distribution of forces applied against the native tissue, which may reduce risks of tissue erosion as a result of pressure from contact. Also, many or most of the diamond-shaped cells of the outer frame 601 may define a smaller area compared to many or most of the cells of the prior art outer frame 101. These smaller diamond-shaped cells may help the outer frame maintain significant stiffness, even at large diameters, which may help resist backpressure during operation and thus resist migration into the left atrium. However, the cells may still allow for collapse of the prosthetic heart valve (including the outer frame 601) into a relatively small delivery system, for example a sheath having a size of about 27 French, without resulting in excessive sheathing forces or strains.

Still further, the relatively large number of cells of outer frame 601 may provide for increased options for locations to attach additional features to the stent. For example, the outer frame 601 may be partially or completely covered by a skirt, such as a fabric skirt, to assist in sealing between the outer fame 601 and the native valve annulus upon implantation. It may be desirable to include additional sealing features, for example extra layers of fabric at strategic locations to even further mitigate blood leaking around the outside of the outer frame 601. The additional cells of outer frame 601 may provide for a relatively large number of options for attaching such additional sealing features to the outer frame 601 in desired locations. Also, although skirt S is illustrated in FIG. 3F as fully covering the outer frame, it may be desirable to cover less than the complete outer frame. For example, the skirt S may cover all of the cells of the outer frame 601, with the exception of the ventricular row 611c, which may result in a generally scalloped outflow edge of the skirt S. By leaving only the ventricular row 611c uncovered by a skirt S, the skirt S may still provide good sealing with the native valve annulus, but blood may flow through the open cells in the ventricular row 611c to help minimize or eliminate LVOT obstruction. A further benefit is that additional attachment options may help provide uniform folding of the fabric during sheathing such that additional intervention (e.g. manual pleating of fabric) is not necessary. An example of uniform stent collapse is shown in FIG. 8A, which illustrates MicroCT images of a prosthetic heart valve incorporating outer frame 601 and inner frame 605 (with only the metallic components having high visibility). Each image in FIG. 8A illustrates the prosthetic heart valve collapsed within the sheath of a delivery device, with each image being taken at 90 degree rotations relative to one another. It can be seen in FIG. 8A that the shape of the collapsed prosthetic heart valve is substantially identical in the different rotational views, illustrating the high uniformity of the shape of the prosthetic heart valve when maintained in the delivery condition.

It should be understood that various different inner frames and outer frames of a prosthetic heart valve are described herein. While certain inner frames are described in connection with other corresponding outer frames, it should be understood that, generally, the different inner frames may be used with the different outer frames as appropriate, particularly when the coupling arms of the inner and outer frames are structured and/or shape-set to mate with each other in a desired configuration. Further, it should be understood that features described in combination with one inner frame (or one outer frame) may be combined with features described for other inner frames (or outer frames) herein. Still further, although generally described for use in replacing a native mitral valve, the prosthetic heart valves described herein may be suitable for use in replacing native tricuspid valves.

According to one aspect of the disclosure, a prosthetic heart valve comprises:
a collapsible and expandable outer frame configured to engage tissue of a native heart valve annulus, the outer frame having an atrial portion adapted to be positioned on an atrial side of the native heart valve annulus, a ventricle portion adapted to be positioned on a ventricle side of the native heart valve annulus, a narrowed waist portion between the atrial portion and the ventricle portion, and a plurality of outer coupling arms having a first end coupled to the outer frame and a second free end, the first ends of the outer coupling arms coupled to the outer frame at a location substantially equidistant between an inflow end of the outer frame and an outflow end of the outer frame;
a collapsible and expandable inner frame positioned radially inward of the outer frame, the inner frame including a plurality of inner coupling arms having a first end coupled to the inner frame and a second free end; and
a prosthetic valve assembly coupled to, and positioned radially inward of, the inner frame;
wherein the second free ends of the outer coupling arms are coupled to the second free ends of the inner coupling arms to couple the outer frame to the inner frame; and/or
the outer coupling arms are integral with the outer frame, and the inner coupling arms are integral with the inner frame; and/or
the second free ends of the outer coupling arms are coupled to the second free ends of the inner coupling arms via mechanical fasteners; and/or
the mechanical fasteners are sutures; and/or
in an expanded condition of the outer frame, the outer coupling arms are contoured so that the second free ends of the outer coupling arms are substantially parallel to a central longitudinal axis of the outer frame, the central longitudinal axis extending from the inflow end of the outer frame to the outflow end of the outer frame; and/or
in an expanded condition of the inner frame, the inner coupling arms are contoured so that the second free ends of the inner coupling arms are substantially parallel to a central longitudinal axis of the inner frame, the central longitudinal axis extending from an inflow end of the inner frame to an outflow end of the inner frame; and/or
the inner frame includes a plurality of rows of substantially diamond-shaped cells, including a first row at the inflow end of the inner frame; and/or
in a collapsed condition of the inner frame, each inner coupling arm is positioned within one of the cells in the first row of cells at the inflow end of the inner frame.

According to another aspect of the disclosure, a prosthetic heart valve comprises:
a collapsible and expandable outer frame configured to engage tissue of a native heart valve annulus, the outer frame having an atrial portion adapted to be positioned on an atrial side of the native heart valve annulus, a ventricle portion adapted to be positioned on a ventricle side of the native heart valve annulus, a narrowed waist portion between the atrial portion and the ventricle portion, and a plurality of outer coupling arms having a first end coupled to the outer frame and a second free end;
a collapsible and expandable inner frame positioned radially inward of the outer frame, the inner frame having a first row of generally diamond shaped cells at an inflow end of the inner frame, and a second row of generally diamond shaped cells at an outflow end of the inner frame, the inner frame including a plurality of inner coupling arms having a first end coupled to the inner frame and a second free end, the second free ends of the outer coupling arms being coupled to the second free ends of the inner coupling arms to couple the outer frame to the inner frame; and
a prosthetic valve assembly coupled to, and positioned radially inward of, the inner frame;
wherein the inner frame includes a plurality of axial struts extending from the second row of generally diamond shaped cells in a direction away from the inflow end of the inner frame, the axial struts defining commissure windows, prosthetic leaflets of the prosthetic valve assembly being coupled to the axial struts via the commissure windows, a plurality of support struts coupling the axial struts to the second row of generally diamond shaped cells; and/or
each axial strut includes a pair of the support struts coupling the axial strut to the second row of generally diamond shaped cells; and/or
each support strut in the pair of support struts has a first end coupled to a terminal end of the axial strut, and a second end coupled to the second row of generally diamond shaped cells; and/or
each support strut in the pair of support struts extends from the axial strut in opposite circumferential directions; and/or
the pair of the support struts and the axial strut together present a blunted atraumatic surface; and/or
each axial strut is coupled to the second row of generally diamond shaped cells at a location between two adjacent cells in the second row of generally diamond shaped cells; and/or
each axial strut is coupled to the second row of generally diamond shaped cells at an outflow apex of a cell in the second row of generally diamond shaped cells.

According to yet another aspect of the disclosure, a prosthetic heart valve comprises:
a collapsible and expandable outer frame configured to engage tissue of a native heart valve annulus, the outer frame having an atrial portion adapted to be positioned on an atrial side of the native heart valve annulus, a ventricle portion adapted to be positioned on a ventricle side of the native heart valve annulus, a narrowed waist portion between the atrial portion and the ventricle portion, and a plurality of outer coupling arms having a first end coupled to the outer frame and a second free end;
a collapsible and expandable inner frame positioned radially inward of the outer frame, the inner frame including a plurality of inner coupling arms having a first end coupled to the inner frame and a second free end, the second free ends of the outer coupling arms being coupled to the second free ends of the inner coupling arms to couple the outer frame to the inner frame; and
a prosthetic valve assembly coupled to, and positioned radially inward of, the inner frame;
wherein the outer frame includes a first row of cells at an inflow end of the outer frame, and a second row of cells at an outflow end of the outer frame, the first row of cells and the second row of cells having the same number of cells; and/or
the first row of cells and the second row of cells each has twenty-four cells; and/or
the first row of cells includes a first type of cell alternating with a second type of cell, the first and second type of cells both being generally diamond shaped, the first type of cell being longer in an axial direction than the second type of cell; and/or
the outer frame includes a pin (or tab) extending from an inflow apex of each of the first type of cell in the first row of cells in a direction toward the outflow end of the outer frame; and/or
in a collapsed condition of the outer frame, each of the first type of cell in the first row of cells has a first width in the circumferential direction adjacent the pin (or tab), and a second width in the circumferential direction at a location spaced away from the pin (or tab), the first width being greater than the second width.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve comprising:
a collapsible and expandable outer frame configured to engage tissue of a native heart valve annulus, the outer frame having an atrial portion adapted to be positioned on an atrial side of the native heart valve annulus, a ventricle portion adapted to be positioned on a ventricle side of the native heart valve annulus, a narrowed waist portion between the atrial portion and the ventricle portion, and a plurality of outer coupling arms having a first end coupled to the outer frame and a second free end, the first ends of the outer coupling arms coupled to the outer frame at a location substantially equidistant between an inflow end of the outer frame and an outflow end of the outer frame;
a collapsible and expandable inner frame positioned radially inward of the outer frame, the inner frame including a plurality of inner coupling arms having a first end coupled to the inner frame and a second free end; and
a prosthetic valve assembly coupled to, and positioned radially inward of, the inner frame;
wherein the second free ends of the outer coupling arms are coupled to the second free ends of the inner coupling arms to couple the outer frame to the inner frame.

2. The prosthetic heart valve of claim 1, wherein the outer coupling arms are integral with the outer frame, and the inner coupling arms are integral with the inner frame.

3. The prosthetic heart valve of claim 1, wherein the second free ends of the outer coupling arms are coupled to the second free ends of the inner coupling arms via mechanical fasteners.

4. The prosthetic heart valve of claim 3, wherein the mechanical fasteners are sutures.

5. The prosthetic heart valve of claim 1, wherein, in an expanded condition of the outer frame, the outer coupling arms are contoured so that the second free ends of the outer coupling arms are substantially parallel to a central longitudinal axis of the outer frame, the central longitudinal axis extending from the inflow end of the outer frame to the outflow end of the outer frame.

6. The prosthetic heart valve of claim 1, wherein, in an expanded condition of the inner frame, the inner coupling arms are contoured so that the second free ends of the inner coupling arms are substantially parallel to a central longitudinal axis of the inner frame, the central longitudinal axis extending from an inflow end of the inner frame to an outflow end of the inner frame.

7. The prosthetic heart valve of claim 1, wherein the inner frame includes a plurality of rows of substantially diamond-shaped cells, including a first row at the inflow end of the inner frame.

8. The prosthetic heart valve of claim 7, wherein in a collapsed condition of the inner frame, each inner coupling arm is positioned within one of the cells in the first row of cells at the inflow end of the inner frame.

9. A prosthetic heart valve comprising:
a collapsible and expandable outer frame configured to engage tissue of a native heart valve annulus, the outer frame having an atrial portion adapted to be positioned on an atrial side of the native heart valve annulus, a ventricle portion adapted to be positioned on a ventricle side of the native heart valve annulus, a narrowed waist portion between the atrial portion and the ventricle portion, and a plurality of outer coupling arms having a first end coupled to the outer frame and a second free end;
a collapsible and expandable inner frame positioned radially inward of the outer frame, the inner frame having a first row of generally diamond shaped cells at an inflow end of the inner frame, and a second row of generally diamond shaped cells at an outflow end of the inner frame, the inner frame including a plurality of inner coupling arms having a first end coupled to the inner frame and a second free end, the second free ends of the outer coupling arms being coupled to the second free ends of the inner coupling arms to couple the outer frame to the inner frame; and
a prosthetic valve assembly coupled to, and positioned radially inward of, the inner frame;
wherein the inner frame includes a plurality of axial struts extending from the second row of generally diamond shaped cells in a direction away from the inflow end of the inner frame, the axial struts defining commissure windows, prosthetic leaflets of the prosthetic valve assembly being coupled to the axial struts via the commissure windows, a plurality of support struts coupling the axial struts to the second row of generally diamond shaped cells.

10. The prosthetic heart valve of claim 9, wherein each axial strut includes a pair of the support struts coupling the axial strut to the second row of generally diamond shaped cells.

11. The prosthetic heart valve of claim 10, wherein each support strut in the pair of support struts has a first end coupled to a terminal end of the axial strut, and a second end coupled to the second row of generally diamond shaped cells.

12. The prosthetic heart valve of claim 10, wherein each support strut in the pair of support struts extends from the axial strut in opposite circumferential directions.

13. The prosthetic heart valve of claim 10, wherein the pair of the support struts and the axial strut together present a blunted atraumatic surface.

14. The prosthetic heart valve of claim 9, wherein each axial strut is coupled to the second row of generally diamond shaped cells at a location between two adjacent cells in the second row of generally diamond shaped cells.

15. The prosthetic heart valve of claim 9, wherein each axial strut is coupled to the second row of generally diamond shaped cells at an outflow apex of a cell in the second row of generally diamond shaped cells.

16. A prosthetic heart valve comprising:
a collapsible and expandable outer frame configured to engage tissue of a native heart valve annulus, the outer frame having an atrial portion adapted to be positioned on an atrial side of the native heart valve annulus, a ventricle portion adapted to be positioned on a ventricle side of the native heart valve annulus, a narrowed waist portion between the atrial portion and the ventricle portion, and a plurality of outer coupling arms having a first end coupled to the outer frame and a second free end;
a collapsible and expandable inner frame positioned radially inward of the outer frame, the inner frame including a plurality of inner coupling arms having a first end coupled to the inner frame and a second free end, the second free ends of the outer coupling arms being coupled to the second free ends of the inner coupling arms to couple the outer frame to the inner frame; and
a prosthetic valve assembly coupled to, and positioned radially inward of, the inner frame;
wherein the outer frame includes a first row of cells at an inflow end of the outer frame, and a second row of cells at an outflow end of the outer frame, the first row of cells and the second row of cells having the same number of cells.

17. The prosthetic heart valve of claim 16, wherein the first row of cells and the second row of cells each has twenty-four cells.

18. The prosthetic heart valve of claim 16, wherein the first row of cells includes a first type of cell alternating with a second type of cell, the first and second type of cells both being generally diamond shaped, the first type of cell being longer in an axial direction than the second type of cell.

19. The prosthetic heart valve of claim 18, wherein the outer frame includes a pin extending from an inflow apex of each of the first type of cell in the first row of cells in a direction toward the outflow end of the outer frame.

20. The prosthetic heart valve of claim 19, wherein, in a collapsed condition of the outer frame, each of the first type of cell in the first row of cells has a first width in a circumferential direction adjacent the pin, and a second width in the circumferential direction at a location spaced away from the pin, the first width being greater than the second width.
